(19) United States
(12) Patent Application Publication
(10) Pub. No.: US 2026/0345639 A1
(43) Pub. Date: Nov. 5, 2026

(54) NOVEL RAMP-UP SCHEDULES FOR TREATING B-CELL MALIGNANCIES

(71) Applicant: BeOne Medicines I GmbH, Basel (CH)

(72) Inventors: Priscille Bourquelot, Wilmington, DE (US); Ying Ou, Wilmington, DE (US); Sheel Patel, Wilmington, DE (US); Zhiyu Tang, Wilmington, DE (US); Remus Vezan, Wilmington, DE (US)

(21) Appl. No.: 19/660,570

(22) Filed: Apr. 28, 2026

Related U.S. Application Data
(60) Provisional application No. 63/796,532, filed on Apr. 29, 2025.
(60) Provisional application No. 63/871,860, filed on Aug. 28, 2025.
(60) Provisional application No. 63/929,361, filed on Dec. 2, 2025.

(51) Int. Cl. A61K 31/635; A61K 45/06; A61P 35/00; A61P 35/02

(52) U.S. Cl. CPC A61K 31/635; A61K 45/06; A61P 35/00; A61P 35/02

(57) ABSTRACT

This application discloses a method of treating B-cell malignancies in a patient, which comprises administering to the patient sonrotoclax or a pharmaceutically acceptable salt thereof per a 5-week twice-a-week ramp-up schedule.

### CROSS-REFERENCE TO RELATED APPLICATIONS

This application claims the benefit of priority of U.S. Provisional Application No. 63/796,532, filed Apr. 29, 2025, U.S. Provisional Application No. 63/871,860, filed Aug. 28, 2025, and U.S. Provisional Application No. 63/929,361, filed Dec. 2, 2025, each of which is incorporated by reference herein in its entirety for any purpose.

### FIELD

This application relates to novel ramp-up schedules used in the methods of treating a patient having a B-cell malignancy with a BCL-2 inhibitor.

### BACKGROUND

B-cell malignancies are cancers arising from abnormal B lymphocytes, such as chronic lymphocytic leukemia (CLL) and mantle cell lymphoma (MCL). CLL is the most common leukemia in the Western world and is considered as a treatable but incurable disease. CLL and small lymphocytic lymphoma (SLL) are different forms of the same illness, such that CLL and SLL are often treated in the same way. Despite introduction of novel agents (e.g., Bruton tyrosine kinase (BTK) inhibitors, B-cell lymphoma 2 (BCL-2) inhibitors) that have changed the treatment landscape, unmet medical needs still exist for CLL/SLL patients.

MCL is an uncommon and incurable B-cell malignancy that is currently treated with combinations of cytotoxic chemotherapy and the anti-CD20 antibody rituximab and more recently oral non-chemotherapy agents especially BTK inhibitors. Despite the aggressive measures that can achieve a high response in treatment-naïve MCL patients, most patients experience relapse or refractory disease that ultimately results in poor outcomes, especially in those who have been previously treated with a BTK inhibitor. BCL-2 is a key regulator of apoptosis, aberrantly expressed in many hematologic malignancies including MCL, and plays a critical role in disease resistance to treatment.

Tumor lysis syndrome (TLS) is a potentially life-threatening condition that can occur during or after cancer treatment, especially in rapidly growing tumors, or highly aggressive solid tumors. It results from the sudden release of intracellular contents into the bloodstream due to rapid tumor cell death. TLS is often a medical emergency. The potency of targeted inhibitors such as BCL-2 inhibitors can be associated with a risk of tumor lysis syndrome (TLS) upon initiation of treatment, particularly in CLL and other sensitive disease types, e.g., MCL.

Venetoclax is a BCL-2 inhibitor approved for treating CLL/SLL, but not MCL. Venetoclax has been reported achieving 65% overall response rate in the patients with post-ibrutinib CLL/SLL, and 2 out of 91 patients having laboratory TLS. See Jones, Jeffrey A., et al., The lancet oncology 19.1 (2018): 65-75. Venetoclax has also been reported achieving 55% overall response rate in the patients with relapsed or refractory (R/R) MCL, and 4 out of 20 patients (20%) having laboratory TLS. See Eyre, Toby A. et al., Haematologica. 2019 February; 104(2):e68-e71. A significant challenge associated with current venetoclax treatment is the requirement or recommendation for patient hospitalization and extensive post-dose monitoring, often necessitating 6-8 or 24 hours of observation (See Table 3, VENCLEXTA FDA label dated June 2022); thus, there remains an unmet need for a novel treatment option for B-cell malignancies, such as CLL/SLL and MCL, that is safer, more effective, and/or less burdensome, on patients and/or caregivers.

International publication WO2019/210828, the entire content of which is incorporated herein by reference, disclosed a series of BCL-2 inhibitors, in particularly, 2-((1H-pyrrolo[2,3-b]pyridin-5-yl)oxy)-N-((4-((((1r,4r)-4-hydroxy-4-methylcyclohexyl)methyl)amino)-3-nitrophenyl) sulfonyl)-4-(2-((S)-2-(2-isopropylphenyl) pyrrolidin-1-yl)-7-azaspiro[3.5]nonan-7-yl)benzamide (hereinafter “sonrotoclax”), having the following chemical structure:

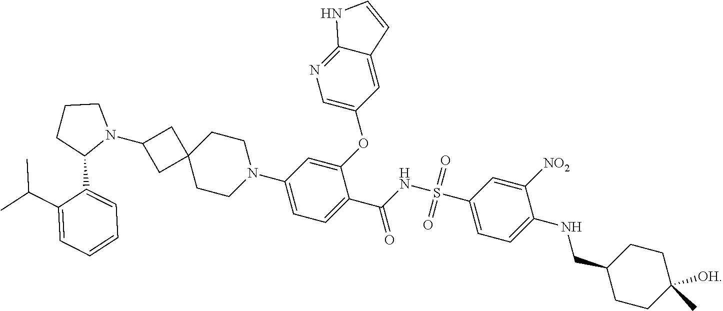 [SMILES: CC(C)c1ccccc1[C@@H]1CCCN1C1CC2(CCN(c3ccc(C(=O)NS(=O)(=O)c4ccc(NC[C@H]5CC[C@](C)(O)CC5)c([N+](=O)[O-])c4)c(Oc4cnc5[nH]ccc5c4)c3)CC2)C1]

### SUMMARY

Provided herein is a method for treating a B-cell malignancy, comprising administering to a patient in need thereof sonrotoclax or a pharmaceutically acceptable salt thereof in therapeutically effective escalating doses followed by a therapeutically effective target daily dose.

Provided herein is also a method for treating a B-cell malignancy while preventing TLS, comprising administering to a patient in need thereof sonrotoclax or a pharmaceutically acceptable salt thereof in therapeutically effective escalating doses followed by a therapeutically effective target daily dose.

Provided herein is also a method for treating a B-cell malignancy while reducing a risk of TLS, comprising administering to a patient in need thereof sonrotoclax or a pharmaceutically acceptable salt thereof in therapeutically effective escalating doses followed by a therapeutically effective target daily dose.

Provided here is also a method for preventing TLS comprising administering to a patient in need thereof sonrotoclax or a pharmaceutically acceptable salt thereof in therapeutically effective escalating doses followed by a therapeutically effective target daily dose, wherein the patient has a B-cell malignancy.

Provided here is also a method for reducing a risk of TLS comprising administering to a patient in need thereof sonrotoclax or a pharmaceutically acceptable salt thereof in therapeutically effective escalating doses followed by a therapeutically effective target daily dose, wherein the patient has a B-cell malignancy.

### BRIEF DESCRIPTION OF FIGURES

FIG. 1 shows the 5-week twice-a-week ramp up schedule until 320 mg per day.

FIG. 2 shows the forest plot for overall response rate by independent review committee (excluding the first 18 patients) as of the first DCO date.

FIG. 3 shows the forest plot for overall response rate by independent review committee subgroup analysis as of the second DCO date.

FIG. 4 shows the forest plot for overall response rate by independent review committee (efficacy analysis set excluding the first 18 patients) as of the second DCO date.

FIG. 5 illustrates the 5-week once-a-week ramp up schedule of sonrotoclax.

FIG. 6 illustrates an alternative 5-week once-a-week ramp up schedule of sonrotoclax.

FIG. 7 illustrates an alternative 5-week once-a-week ramp up schedule of sonrotoclax.

FIG. 8 illustrates an alternative 5-week once-a-week ramp up schedule of sonrotoclax.

FIG. 9 illustrates the study treatment timeline with the 5-week once-a-week ramp up schedule of sonrotoclax.

### DETAILED DESCRIPTION

### I. Definitions

Unless specifically defined elsewhere in this document, all other technical and scientific terms used herein have the meaning commonly understood by one of ordinary skilled in the art.

As used in this specification, the singular forms “a,” “an,” and “the” include plural referents unless the context clearly dictates otherwise.

“About” when used immediately preceding a numerical value means a range of plus or minus 10% of that value, e.g., “about 50 mg” means 45 to 55 mg. When “about” used immediately before a numeric range of values, it means a range from minus 10% of the lower limit to plus 10% of the upper limit. For example, “about 50-600 mg” means 45-660 mg.

“Anti-CD20 based therapy” as used herein refers to an agent that specifically binds to the CD20. Such agents include, but are not limited to, monoclonal antibodies, antibody fragments, bispecific antibodies, antibody-drug conjugates. Exemplary anti-CD20 agents include rituximab, obinutuzumab, ofatumumab, ocrelizumab, ublituximab, and veltuzumab.

“Adverse reaction” as used herein was graded using NCI CTCAE version 5.0 which is the standardized definitions for adverse events (AEs), known as the Common Terminology Criteria for Adverse Events (CTCAE), version 5.0 published in November 2017 by the National Cancer Institute (NCI) of the National Institutes of Health (NIH).

“Antihyperuricemic treatment” as used herein refers to an agent that lowers serum uric acid levels in a subject. Antihyperuricemic agents include, but are not limited to, xanthine oxidase inhibitors (e.g., allopurinol, febuxostat), uricosuric agents (e.g., probenecid, lesinurad), and recombinant uricase enzymes (e.g., pegloticase).

“BTK inhibitor” or “BTKi” as used herein refers to an agent that inhibits the activity of Bruton's tyrosine kinase (BTK). BTK inhibitors include, but are not limited to, covalent inhibitors (e.g., ibrutinib, acalabrutinib, and zanubrutinib) and non-covalent inhibitors (e.g., pirtobrutinib).

“Chemoimmunotherapy” or “CIT” as used herein refers to a therapeutic regimen that combines one or more chemotherapeutic agents with one or more immunotherapeutic agents for the treatment, prevention, or management of a disease, typically cancer. Chemotherapeutic agents include, but are not limited to, alkylating agents, antimetabolites, anthracyclines, plant alkaloids, and platinum-based compounds. Immunotherapeutic agents include, but are not limited to, immune checkpoint inhibitors (e.g., anti-PD-1, anti-PD-L1, anti-CTLA-4 antibodies), cytokines, cancer vaccines, and adoptive cell therapies. Exemplary CIT includes, but not limited to, FCR Regimen (e.g., fludarabine, cyclophosphamide, and rituximab), BR Regimen (e.g., bendamustine and rituximab), Chlorambucil-Based Chemoimmunotherapy (e.g., chlorambucil and obinutuzumab), R-CHOP (e.g., cyclophosphamide, doxorubicin, vincristine, prednisone, and rituximab), R-BAC Regimen (e.g., bendamustine, cytarabine, and rituximab), and HyperCVAD+Rituximab Regimen (e.g., hyper-fractionated cyclophosphamide, vincristine, doxorubicin, dexamethasone, and rituximab).

“Effective” or “therapeutically effective” as used herein means capable of treating or preventing a disorder, disease, condition, or symptoms thereof.

“Half of a week” as used herein refers to 2, 3, 4, or 5 days. In some embodiments, the term “half of a week” as used herein refers to 3 or 4 days. In some embodiments, the term “half of a week” as used herein refers to 3 days. In some embodiments, the term “half of a week” as used herein refers to 4 days.

“Hematological toxicity” as used herein refers to Grade ≥3 febrile neutropenia, Grade ≥3 thrombocytopenia that results in clinically significant bleeding, Grade 4 thrombocytopenia, or any Grade ≥4 hematologic toxicity lasting >7 consecutive days.

“Nonhematological toxicity” as used herein refers to any Grade ≥3 nonhematologic toxicity, but does not include Grade 3 gastrointestinal (GI) toxicity such as nausea, vomiting, diarrhea unless unresponsive to treatment ≥3 days, or asymptomatic biochemical laboratory abnormalities that resolve ≤7 days.

“Overall Response Rate” or “ORR” as used herein refers to the proportion of patients achieving: CR, CRi, nPR, or PR per the 2018 iwCLL Guidelines for patients with CLL, or PR or better per the Lugano Classification for non-hodgkin's lymphoma (NHL) for patients with SLL and MCL. “Response rate” as used herein refers to the likelihood of an individual patient with CLL that will achieve CR, CRi, nPR, or PR per the 2018 iwCLL Guidelines, or the likelihood of an individual patient with SLL or MCL that will achieve PR or better per the Lugano Classification for NHL.

“Patient” as used herein refers to human.

“Pharmaceutically acceptable salt” as used herein refers to a salt form of a compound (e.g., a drug) having at least one group capable for salt formation that causes no significant adverse toxicological effects to the subject. Pharmaceutically acceptable salts include, for example, salts prepared by reaction with an inorganic acid, organic acid, or a base depending on the nature of sonrotoclax (e.g., drug). The inorganic acid can be hydrochloric acid, hydrobromic acid, carbonic acid, sulfuric acid, phosphoric acid, and the like; the organic acid can be fumaric acid, maleic acid, succinic acid, acetic acid, citric acid, tartaric acid, methanesulfonic acid and the like.

“Solvate” as used herein refers to a physical association of sonrotoclax or a pharmaceutically acceptable salt thereof with one or more pharmaceutically acceptable solvent molecules. This physical association involves varying degrees of ionic and covalent bonding, including hydrogen bonding. In some embodiments, solvate means hydrate wherein the pharmaceutically acceptable solvent is H₂O.

Sonrotoclax or a pharmaceutically acceptable salt thereof as used herein include, but are not limited to, their solvate forms.

“Treating”, “treatment”, or “treat” (of) a disease as used herein refers to slowing or arresting the development of a disease, providing relief from the symptoms or side-effects of the disease, and/or causing regression of the disease.

“Tumor Lysis Syndrome” or “TLS” as used herein refers to an oncologic emergency that is caused by rapid tumor cell lysis leading to a release of large amounts of potassium, phosphate, and nucleic acids into systemic circulation. TLS can be separated into laboratory TLS and clinical TLS. Laboratory TLS must have 2 or more metabolic abnormalities present during the 24-hour period within 3 days before the start of therapy or up to 7 days afterward. Clinical TLS requires the presence of laboratory TLS with clinical consequences such as acute kidney injury, seizures, cardiac dysrhythmia, or death.

The daily dose (such as 1 mg, 2 mg, 5 mg, 10 mg, 20 mg, 40 mg, 80 mg, 160 mg, or 320 mg) of sonrotoclax as used herein is measured in the free form of sonrotoclax. As one of ordinary skilled in the art would understand, if sonrotoclax is administered in the form of a pharmaceutically acceptable salt or a solvate, the amount administered daily thereof will be such to afford the equivalent daily dose of sonrotoclax in its free form.

### II. Embodiments

The following embodiments are not intended to be an explicit or implicit admission that these embodiments are independent or distinct nor should it be interpreted as such. Rather, it is intended to convey information so that the full breadth of the present disclosure can be understood. Furthermore, the following embodiments are not meant to be limiting on the full breadth of the disclosure as recited herein. This disclosure intends to provide every combination of technically compatible embodiments, even if they are not expressly disclosed in combination or linked to each other.

Provided herein is also sonrotoclax or a pharmaceutically acceptable salt thereof for use as described herein.

Provided herein is also a pharmaceutical composition comprising sonrotoclax or a pharmaceutically acceptable salt thereof for use as described herein.

In some embodiments, the B-cell malignancy is CLL/SLL or MCL, particularly R/R CLL/SLL or R/R MCL.

In some embodiments, the TLS is clinical TLS. In some embodiments, the TLS is laboratory TLS.

In some embodiments, the escalating doses are effective in preventing TLS. In some embodiments, the escalating doses are effective in preventing clinical TLS. In some embodiments, the escalating doses are effective in preventing laboratory TLS.

In some embodiments, the escalating doses are effective in reducing a risk of TLS. In some embodiments, the escalating doses are effective in reducing a risk of clinical TLS. In some embodiments, the escalating doses are effective in reducing a risk of laboratory TLS.

In some embodiments, the patient is administered with an antihyperuricemic treatment prior to the administration of the first dose of sonrotoclax or a pharmaceutically acceptable salt thereof. In some embodiments, the antihyperuricemic treatment is administered about two to about three days prior to the administration of the first dose of sonrotoclax or a pharmaceutically acceptable salt thereof. In some embodiments, the antihyperuricemic treatment is administered about two to about three days prior to the administration of the first dose of sonrotoclax or a pharmaceutically acceptable salt thereof, and continues until at least 7 days after the dose of sonrotoclax or a pharmaceutically acceptable salt thereof reaches the target daily dose.

In some embodiments, reducing a risk of TLS is compared with treatment with target daily dose of sonrotoclax without escalating doses of sonrotoclax, or compared with another known treatment, particularly another BCL-2 inhibitor, more particularly venetoclax.

Provided herein is also a method for treating a B-cell malignancy comprising (1) administering to a patient in need thereof an antihyperuricemic treatment, and (2) administering to the patient sonrotoclax or a pharmaceutically acceptable salt thereof in therapeutically effective escalating doses followed by a therapeutically effective target daily dose.

Provided herein is also a method for treating a B-cell malignancy while preventing TLS or reducing a risk of TLS, comprising (1) administering to a patient in need thereof an antihyperuricemic treatment, and (2) administering to the patient sonrotoclax or a pharmaceutically acceptable salt thereof in therapeutically effective escalating doses followed by a therapeutically effective target daily dose.

Provided herein is also a method for preventing TLS or reducing the risk of TLS comprising (1) administering to a patient in need thereof an antihyperuricemic treatment, and (2) administering to the patient sonrotoclax or a pharmaceutically acceptable salt thereof in therapeutically effective escalating doses followed by a therapeutically effective target daily dose.

In some embodiments, the therapeutically effective target daily dose is about 320 mg of sonrotoclax. In some embodiments, the therapeutically effective target daily dose is about 160 mg of sonrotoclax. In some embodiments, the therapeutically effective target daily dose is about 640 mg of sonrotoclax.

In some embodiments, the escalating doses comprise one or more doses selected from a dose of about 1 mg of sonrotoclax per day for half of a week, a dose of about 2 mg of sonrotoclax per day for half of a week, a dose of about 5 mg of sonrotoclax per day for half of a week, a dose of about 10 mg of sonrotoclax per day for half of a week, a dose of about 20 mg of sonrotoclax per day for half of a week, a dose of about 40 mg of sonrotoclax per day for half of a week, a dose of about 80 mg of sonrotoclax per day for half of a week, and a dose of about 160 mg of sonrotoclax per day for half of a week.

In some embodiments, the escalating doses comprise a dose of about 1 mg of sonrotoclax per day for half of a week.

In some embodiments, the escalating doses comprise a dose of about 2 mg of sonrotoclax per day for half of a week.

In some embodiments, the escalating doses comprise a dose of about 5 mg of sonrotoclax per day for half of a week.

In some embodiments, the escalating doses comprise a dose of about 10 mg of sonrotoclax per day for half of a week.

In some embodiments, the escalating doses comprise a dose of about 20 mg of sonrotoclax per day for half of a week.

In some embodiments, the escalating doses comprise a dose of about 40 mg of sonrotoclax per day for half of a week.

In some embodiments, the escalating doses comprise a dose of about 80 mg of sonrotoclax per day for half of a week.

In some embodiments, the escalating doses comprise a dose of about 160 mg of sonrotoclax per day for half of a week.

In some embodiments, the escalating doses comprise a dose of about 1 mg of sonrotoclax per day for half of a week, followed by a dose of about 2 mg of sonrotoclax per day for half of a week.

In some embodiments, the escalating doses comprise a dose of about 1 mg of sonrotoclax per day for half of a week, followed by a dose of about 5 mg of sonrotoclax per day for half of a week.

In some embodiments, the escalating doses comprise a dose of about 2 mg of sonrotoclax per day for half of a week, followed by a dose of about 5 mg of sonrotoclax per day for half of a week.

In some embodiments, the escalating doses comprise a dose of about 1 mg of sonrotoclax per day for half of a week, followed by a dose of about 2 mg of sonrotoclax per day for half of a week, and followed by a dose of 5 mg of sonrotoclax per day for half of the week.

In some embodiments, the escalating doses comprise a dose of about 1 mg of sonrotoclax per day for half of a week, followed by a dose of about 2 mg of sonrotoclax per day for half of a week, followed by a dose of about 5 mg of sonrotoclax per day for half of a week, and followed by a dose of about 10 mg of sonrotoclax per day for half of a week.

In some embodiments, the escalating doses comprise a dose of about 1 mg of sonrotoclax per day for half of a week, followed by a dose of about 2 mg of sonrotoclax per day for half of a week, followed by a dose of about 5 mg of sonrotoclax per day for half of a week, followed by a dose of about 10 mg of sonrotoclax per day for half of a week, and followed by a dose of about 20 mg of sonrotoclax per day for half of a week.

In some embodiments, the escalating doses comprise a dose of about 1 mg of sonrotoclax per day for half of a week, followed by a dose of about 2 mg of sonrotoclax per day for half of a week, followed by a dose of about 5 mg of sonrotoclax per day for half of a week, followed by a dose of about 10 mg of sonrotoclax per day for half of a week, followed by a dose of about 20 mg of sonrotoclax per day for half of a week, and followed by a dose of about 40 mg of sonrotoclax per day for half of a week.

In some embodiments, the escalating doses comprise a dose of about 1 mg of sonrotoclax per day for half of a week, followed by a dose of about 2 mg of sonrotoclax per day for half of a week, followed by a dose of about 5 mg of sonrotoclax per day for half of a week, followed by a dose of about 10 mg of sonrotoclax per day for half of a week, followed by a dose of about 20 mg of sonrotoclax per day for half of a week, followed by a dose of about 40 mg of sonrotoclax per day for half of a week, and followed by a dose of about 80 mg of sonrotoclax per day for half of a week.

In some embodiments, the escalating doses comprise a dose of about 1 mg of sonrotoclax per day for half of a week, followed by a dose of about 2 mg of sonrotoclax per day for half of a week, followed by a dose of about 5 mg of sonrotoclax per day for half of a week, followed by a dose of about 10 mg of sonrotoclax per day for half of a week, followed by a dose of about 20 mg of sonrotoclax per day for half of a week, followed by a dose of about 40 mg of sonrotoclax per day for half of a week, followed by a dose of about 80 mg of sonrotoclax per day for half of a week, and followed by a dose of about 160 mg of sonrotoclax per day for half of a week.

In some embodiments, the escalating doses comprise a dose of about 80 mg of sonrotoclax per day for half of a week, and followed by a dose of about 160 mg of sonrotoclax per day for half a week.

In some embodiments, the escalating doses comprise a dose of about 40 mg of sonrotoclax per day for half of a week, followed by a dose of about 80 mg of sonrotoclax per day for half of a week, and followed by a dose of about 160 mg of sonrotoclax per day for half a week.

In some embodiments, the escalating doses comprise a dose of about 20 mg of sonrotoclax per day for half of a week, followed by a dose of about 40 mg of sonrotoclax per day for half of a week, followed by a dose of about 80 mg of sonrotoclax per day for half of a week, and followed by a dose of about 160 mg of sonrotoclax per day for half a week.

In some embodiments, the escalating doses comprise a dose of about 10 mg of sonrotoclax per day for half of a week, followed by a dose of about 20 mg of sonrotoclax per day for half of a week, followed by a dose of about 40 mg of sonrotoclax per day for half of a week, followed by a dose of about 80 mg of sonrotoclax per day for half of a week, and followed by a dose of about 160 mg of sonrotoclax per day for half a week.

In some embodiments, the escalating doses comprise a dose of about 5 mg of sonrotoclax per day for half of a week, followed by a dose of about 10 mg of sonrotoclax per day for half of a week, followed by a dose of about 20 mg of sonrotoclax per day for half of a week, followed by a dose of about 40 mg of sonrotoclax per day for half of a week, followed by a dose of about 80 mg of sonrotoclax per day for half of a week, and followed by a dose of about 160 mg of sonrotoclax per day for half a week.

In some embodiments, the escalating doses comprise a dose of about 2 mg of sonrotoclax per day for half of a week, followed by a dose of about 5 mg of sonrotoclax per day for half of a week, followed by a dose of about 10 mg of sonrotoclax per day for half of a week, followed by a dose of about 20 mg of sonrotoclax per day for half of a week, followed by a dose of about 40 mg of sonrotoclax per day for half of a week, followed by a dose of about 80 mg of sonrotoclax per day for half of a week, and followed by a dose of about 160 mg of sonrotoclax per day for half a week.

In some embodiments, the escalating doses comprise a dose of about 1 mg of sonrotoclax per day for half of a week, followed by a dose of about 2 mg of sonrotoclax per day for half a week, followed by a dose of about 5 mg of sonrotoclax per day for half of a week, followed by a dose of about 10 mg of sonrotoclax per day for half of a week, followed by a dose of about 20 mg of sonrotoclax per day for half of a week, followed by a dose of about 40 mg of sonrotoclax per day for half of a week, followed by a dose of about 80 mg of sonrotoclax per day for half of a week, and followed by a dose of about 160 mg of sonrotoclax per day for half of a week, and the escalating doses are followed by a therapeutically effective target daily dose of about 320 mg of sonrotoclax.

In some embodiments, half of week is 2 days.

In some embodiments, half of week is 3 days.

In some embodiments, half of week is 4 days.

In some embodiments, half of week is 5 days.

In some embodiments, the escalating doses comprise a dose of about 160 mg of sonrotoclax per day for four days.

In some embodiments, the escalating doses comprise a dose of about 80 mg of sonrotoclax per day for three days, and followed by a dose of about 160 mg of sonrotoclax per day for four days.

In some embodiments, the escalating doses comprise a dose of about 40 mg of sonrotoclax per day for four days, followed by a dose of about 80 mg of sonrotoclax per day for three days, and followed by a dose of about 160 mg of sonrotoclax per day for four days.

In some embodiments, the escalating doses comprise a dose of about 20 mg of sonrotoclax per day for three days, followed by a dose of about 40 mg of sonrotoclax per day for four days, followed by a dose of about 80 mg of sonrotoclax per day for three days, and followed by a dose of about 160 mg of sonrotoclax per day for four days.

In some embodiments, the escalating doses comprise a dose of about 10 mg of sonrotoclax per day for four days, followed by a dose of about 20 mg of sonrotoclax per day for three days, followed by a dose of about 40 mg of sonrotoclax per day for four days, followed by a dose of about 80 mg of sonrotoclax per day for three days, and followed by a dose of about 160 mg of sonrotoclax per day for four days.

In some embodiments, the escalating doses comprise a dose of about 5 mg of sonrotoclax per day for three days, followed by a dose of about 10 mg of sonrotoclax per day for four days, followed by a dose of about 20 mg of sonrotoclax per day for three days, followed by a dose of about 40 mg of sonrotoclax per day for four days, followed by a dose of about 80 mg of sonrotoclax per day for three days, and followed by a dose of about 160 mg of sonrotoclax per day for four days.

In some embodiments, the escalating doses comprise a dose of about 2 mg of sonrotoclax per day for four days, followed by a dose of about 5 mg of sonrotoclax per day for three days, followed by a dose of about 10 mg of sonrotoclax per day for four days, followed by a dose of about 20 mg of sonrotoclax per day for three days, followed by a dose of about 40 mg of sonrotoclax per day for four days, followed by a dose of about 80 mg of sonrotoclax per day for three days, and followed by a dose of about 160 mg of sonrotoclax per day for four days. In some embodiments, the escalating doses comprise a dose of about 1 mg of sonrotoclax per day for three days.

In some embodiments, the escalating doses comprise a dose of about 1 mg of sonrotoclax per day for three days.

In some embodiments, the escalating doses comprise a dose of about 1 mg of sonrotoclax per day for three days, followed by a dose of about 2 mg of sonrotoclax per day from four days.

In some embodiments, the escalating doses comprise a dose of about 1 mg of sonrotoclax per day for three days, followed by a dose of about 2 mg of sonrotoclax per day for four days, and followed by a dose of about 5 mg of sonrotoclax per day for three days.

In some embodiments, the escalating doses comprise a dose of about 1 mg of sonrotoclax per day for three days, followed by a dose of about 2 mg of sonrotoclax per day for four days, followed by a dose of about 5 mg of sonrotoclax per day for three days, and followed by a dose of about 10 mg of sonrotoclax per day for four days.

In some embodiments, the escalating doses comprise a dose of about 1 mg of sonrotoclax per day for three days, followed by a dose of about 2 mg of sonrotoclax per day for four days, followed by a dose of about 5 mg of sonrotoclax per day for three days, and followed by a dose of about 10 mg of sonrotoclax per day for four days, followed by a dose of about 20 mg of sonrotoclax per day for three days.

In some embodiments, the escalating doses comprise a dose of about 1 mg of sonrotoclax per day for three days, followed by a dose of about 2 mg of sonrotoclax per day for four days, followed by a dose of about 5 mg of sonrotoclax per day for three days, followed by a dose of about 10 mg of sonrotoclax per day for four days, followed by a dose of about 20 mg of sonrotoclax per day for three days, and followed by a dose of about 40 mg of sonrotoclax per day for four days.

In some embodiments, the escalating doses comprise a dose of about 1 mg of sonrotoclax per day for three days, followed by a dose of about 2 mg of sonrotoclax per day for four days, followed by a dose of about 5 mg of sonrotoclax per day for three days, followed by a dose of about 10 mg of sonrotoclax per day for four days, followed by a dose of about 20 mg of sonrotoclax per day for three days, followed by a dose of about 40 mg of sonrotoclax per day for four days, and followed by a dose of about 80 mg of sonrotoclax per day for three days.

In some embodiments, the escalating doses comprise a dose of about 1 mg of sonrotoclax per day for three days, followed by a dose of about 2 mg of sonrotoclax per day for four days, followed by a dose of about 5 mg of sonrotoclax per day for three days, followed by a dose of about 10 mg of sonrotoclax per day for four days, followed by a dose of about 20 mg of sonrotoclax per day for three days, followed by a dose of about 40 mg of sonrotoclax per day for four days, followed by a dose of about 80 mg of sonrotoclax per day for three days, and followed by a dose of about 160 mg of sonrotoclax per day for four days.

In some embodiments, the escalating doses comprise a dose of about 1 mg of sonrotoclax per day from Week 1 Day 1 to Week 1 Day 3, followed by a dose of about 2 mg of sonrotoclax per day from Week 1 Day 4 to Week 1 Day 7, followed by a dose of about 5 mg of sonrotoclax per day from Week 2 Day 1 to Week 2 Day 3, followed by a dose of about 10 mg of sonrotoclax per day from Week 2 Day 4 to Week 2 Day 7, followed by a dose of about 20 mg of sonrotoclax per day from Week 3 Day 1 to Week 3 Day 3, and followed by a dose of about 40 mg of sonrotoclax per day from Week 3 Day 4 to Week 3 Day 7, followed by a dose of about 80 mg of sonrotoclax per day from Week 4 Day 1 to Week 4 Day 3, and followed by a dose of about 160 mg of sonrotoclax per day from Week 4 Day 4 to Week 4 Day 7.

In some embodiments, the escalating doses comprise a dose of about 1 mg of sonrotoclax per day from Week 1 Day 1 to Week 1 Day 3, followed by a dose of about 2 mg of sonrotoclax per day from Week 1 Day 4 to Week 1 Day 7, followed by a dose of about 5 mg of sonrotoclax per day from Week 2 Day 1 to Week 2 Day 3, followed by a dose of about 10 mg of sonrotoclax per day from Week 2 Day 4 to Week 2 Day 7, followed by a dose of about 20 mg of sonrotoclax per day from Week 3 Day 1 to Week 3 Day 3, followed by a dose of about 40 mg of sonrotoclax per day from Week 3 Day 4 to Week 3 Day 7, followed by a dose of about 80 mg of sonrotoclax per day from Week 4 Day 1 to Week 4 Day 3, and followed by a dose of about 160 mg of sonrotoclax per day from Week 4 Day 4 to Week 4 Day 7, and the escalating doses are followed by a therapeutically effective target daily dose of about 320 mg of sonrotoclax starting from Week 5 Day 1.

In some embodiments, the escalating doses comprise a dose of about 1 mg of sonrotoclax per day from Day 1 to 3, followed by a dose of about 2 mg of sonrotoclax per day from Day 4 to 7, followed by a dose of about 5 mg of sonrotoclax per day from Day 8 to 10, followed by a dose of about 10 mg of sonrotoclax per day from Day 11 to 14, followed by a dose of about 20 mg of sonrotoclax per day from Day 15 to 17, followed by a dose of about 40 mg of sonrotoclax per day from Day 18 to 21, followed by a dose of about 80 mg of sonrotoclax per day from Day 22 to 24, followed by a dose of about 160 mg of sonrotoclax per day from Day 25 to 28, and the escalating doses are followed by a therapeutically effective target daily dose of about 320 mg of sonrotoclax starting from Day 29.

In some embodiments, the escalating dose comprise a dose of about 1 mg of sonrotoclax per day for the first week, followed by a dose of about 2 mg of sonrotoclax per day for the second week, followed by a dose of about 5 mg of sonrotoclax per day for the third week, followed by a dose of about 10 mg of sonrotoclax per day for the fourth week, followed by a dose of about 20 mg of sonrotoclax per day for the fifth week, followed by a dose of about 40 mg of sonrotoclax per day for the sixth week, followed by a dose of about 80 mg of sonrotoclax per day for the seventh week, and followed by a dose of about 160 mg of sonrotoclax per day for the eighth week.

In some embodiments, the escalating dose comprise a dose of about 1 mg of sonrotoclax per day for the first week, followed by a dose of about 2 mg of sonrotoclax per day for the second week, followed by a dose of about 5 mg of sonrotoclax per day for the third week, followed by a dose of about 10 mg of sonrotoclax per day for the fourth week, followed by a dose of about 20 mg of sonrotoclax per day for the fifth week, followed by a dose of about 40 mg of sonrotoclax per day for the sixth week, followed by a dose of about 80 mg of sonrotoclax per day for the seventh week, followed by a dose of about 160 mg of sonrotoclax per day for the eighth week, and followed by a therapeutically effective target daily dose of about 320 mg of sonrotoclax.

In some embodiments, sonrotoclax is administered once per day.

In some embodiments, the B-cell malignancy is CLL, SLL, or MCL.

In some embodiments, the B-cell malignancy is R/R CLL.

In some embodiments, the B-cell malignancy is R/R SLL.

In some embodiments, the patient is an adult patient having CLL/SLL, particularly R/R CLL/SLL, who has received at least one treatment for CLL/SLL previously. In some embodiments, the patient is an adult patient having CLL/SLL, particularly R/R CLL/SLL, who has received at least one BTK inhibitor (BTKi) therapy previously. In some embodiments, the patient is an adult patient having CLL/SLL, particularly R/R CLL/SLL, who has received at least one chemoimmunotherapy (CIT) previously. In some embodiments, the patient is an adult patient having CLL/SLL, particularly R/R CLL/SLL, who has received at least one BTKi therapy and one CIT previously. In some embodiments, the patient is an adult patient having CLL/SLL, particularly R/R CLL/SLL, who has received one BTKi therapy previously and is ineligible or intolerant to CIT. In some embodiments, the patient is an adult patient having CLL/SLL who has: (a) treatment intolerance or failure during or after treatment with at least one CIT and at least one BTKi; or (b) treatment intolerance or failure during or after treatment with at least one BTKi and is CIT ineligible. In some embodiments, the BTKi is a covalent or non-covalent BTKi.

In one embodiment, the patient is an adult who has been previously treated for chronic lymphocytic leukemia (CLL), small lymphocytic lymphoma (SLL), or mantle cell lymphoma (MCL).

In one embodiment, the patient is an adult who has been previously treated with a BTK inhibitor. In one embodiment, the patient is an adult who has been previously treated with a BTK inhibitor and anti-CD20 based therapy. In one embodiment, the patient is an adult who has been previously treated with a covalent BTK inhibitor and anti-CD20 based therapy. In one embodiment, the patient is an adult who has been previously treated with a non-covalent BTK inhibitor and anti-CD20 based therapy.

In one embodiment, the patient is an adult who has been previously treated with a BCL-2 inhibitor. In one embodiment, the patient is an adult who has been previously treated with a BCL-2 inhibitor and anti-CD20 based therapy.

In one embodiment, the patient is an adult who has del (17p) or TP53 mutation. In one embodiment, the patient is an adult who has del (17p) mutation. In one embodiment, the patient is an adult who has TP53 mutation.

In some embodiments, the B-cell malignancy is R/R MCL.

In some embodiments, the patient is an adult patient having MCL, particularly R/R MCL, and has received at least one anti-CD20-based therapy previously. In some embodiments, the patient is an adult patient having MCL, particularly R/R MCL, and has received at least one BTKi therapy previously. In some embodiments, the patient is an adult patient having MCL, particularly R/R MCL, and has received at least one anti-CD20-based therapy and one BTKi therapy previously. In some embodiments, the patient is an adult patient having MCL, particularly R/R MCL, who has received BTKi therapy and is ineligible or intolerant to anti-CD20-based therapy. In some embodiments, BTKi therapy is a covalent or non-covalent BTKi therapy.

In some embodiments, the B-cell malignancy is treatment-naïve CLL.

In some embodiments, the B-cell malignancy is treatment-naïve SLL.

In some embodiments, the B-cell malignancy is treatment-naïve MCL.

In some embodiments, the administration of sonrotoclax or a pharmaceutically acceptable salt thereof is discontinued if the patient has undetectable minimal residual disease at less than 10⁻⁴ sensitivity (uMRD4) rate.

In some embodiments, the administration of sonrotoclax or a pharmaceutically acceptable salt thereof is discontinued if the patient has undetectable minimal residual disease at less than 10⁻⁶ sensitivity (uMRD6) rate.

In some embodiments, sonrotoclax is administered orally.

In some embodiments, sonrotoclax is administered with food.

Provided herein is also a method for increasing response rate, comprising administering to a patient in need thereof sonrotoclax or a pharmaceutically acceptable salt thereof in therapeutically effective escalating doses followed by a therapeutically effective target daily dose, wherein the patient has a B-cell malignancy, particularly CLL/SLL or MCL, more particularly R/R CLL/SLL or R/R MCL.

Provided herein is also a method for increasing ORR, comprising administering to patients in need thereof sonrotoclax or a pharmaceutically acceptable salt thereof in therapeutically effective escalating doses followed by a therapeutically effective target daily dose, wherein the patients have a B-cell malignancy, particularly CLL/SLL or MCL, more particularly R/R CLL/SLL or R/R MCL.

In some embodiments, increasing response rate or ORR is compared with no treatment or with another known treatment, particularly another BCL-2 inhibitor, more particularly venetoclax.

Provided here is also a method for improving response rate while preventing or reducing a risk of TLS, comprising administering to a patient in need thereof sonrotoclax or a pharmaceutically acceptable salt thereof in therapeutically effective escalating doses followed by a therapeutically effective target daily dose, wherein the patient has a B-cell malignancy, particularly CLL/SLL or MCL, more particularly R/R CLL/SLL or R/R MCL.

Provided here is also a method for improving ORR while preventing or reducing a risk of TLS, comprising administering to patients in need thereof sonrotoclax or a pharmaceutically acceptable salt thereof in therapeutically effective escalating doses followed by a therapeutically effective target daily dose, wherein the patient has a B-cell malignancy, particularly CLL/SLL or MCL, more particularly R/R CLL/SLL or R/R MCL.

Provided herein is also a method for treating a B-cell malignancy, particularly CLL/SLL or MCL, more particularly R/R CLL, R/R SLL, or R/R MCL comprising administering to a patient in need thereof sonrotoclax or a pharmaceutically acceptable salt thereof in therapeutically effective escalating doses followed by a therapeutically effective target daily dose, wherein the escalating doses comprise a dose of about 1 mg of sonrotoclax per day from Day 1 to 3, followed by a dose of about 2 mg of sonrotoclax per day from Day 4 to 7, followed by a dose of about 5 mg of sonrotoclax per day from Day 8 to 10, followed by a dose of about 10 mg of sonrotoclax per day from Day 11 to 14, followed by a dose of about 20 mg of sonrotoclax per day from Day 15 to 17, followed by a dose of about 40 mg of sonrotoclax per day from Day 18 to 21, followed by a dose of about 80 mg of sonrotoclax per day from Day 22 to 24, followed by a dose of about 160 mg of sonrotoclax per day from Day 25 to 28, and the therapeutically effective target daily dose is about 320 mg of sonrotoclax and administered starting from Day 29.

Provided herein is also a method for treating a B-cell malignancy, particularly CLL/SLL or MCL, more particularly R/R CLL, R/R SLL, or R/R MCL while preventing TLS or reducing the risk of TLS comprising administering to a patient in need thereof sonrotoclax or a pharmaceutically acceptable salt thereof in therapeutically effective escalating doses followed by a therapeutically effective target daily dose, wherein the escalating doses comprise a dose of about 1 mg of sonrotoclax per day from Day 1 to 3, followed by a dose of about 2 mg of sonrotoclax per day from Day 4 to 7, followed by a dose of about 5 mg of sonrotoclax per day from Day 8 to 10, followed by a dose of about 10 mg of sonrotoclax per day from Day 11 to 14, followed by a dose of about 20 mg of sonrotoclax per day from Day 15 to 17, followed by a dose of about 40 mg of sonrotoclax per day from Day 18 to 21, followed by a dose of about 80 mg of sonrotoclax per day from Day 22 to 24, followed by a dose of about 160 mg of sonrotoclax per day from Day 25 to 28, and the therapeutically effective target daily dose is about 320 mg of sonrotoclax and administered starting from Day 29.

Provided herein is also a method for preventing TLS or reducing the risk of TLS comprising administering to a patient in need thereof sonrotoclax or a pharmaceutically acceptable salt thereof in therapeutically effective escalating doses followed by a therapeutically effective target daily dose, wherein the escalating doses comprise a dose of about 1 mg of sonrotoclax per day from Day 1 to 3, followed by a dose of about 2 mg of sonrotoclax per day from Day 4 to 7, followed by a dose of about 5 mg of sonrotoclax per day from Day 8 to 10, followed by a dose of about 10 mg of sonrotoclax per day from Day 11 to 14, followed by a dose of about 20 mg of sonrotoclax per day from Day 15 to 17, followed by a dose of about 40 mg of sonrotoclax per day from Day 18 to 21, followed by a dose of about 80 mg of sonrotoclax per day from Day 22 to 24, followed by a dose of about 160 mg of sonrotoclax per day from Day 25 to 28, and the therapeutically effective target daily dose is about 320 mg of sonrotoclax and administered starting from Day 29, and wherein the patient has a B-cell malignancy, particularly CLL/SLL or MCL, more particularly R/R CLL/SLL or R/R MCL.

Provided herein is also a method for increasing response rate comprising administering to a patient in need thereof sonrotoclax or a pharmaceutically acceptable salt thereof in therapeutically effective escalating doses, wherein the escalating doses comprise a dose of about 1 mg of sonrotoclax per day from Day 1 to 3, followed by a dose of about 2 mg of sonrotoclax per day from Day 4 to 7, followed by a dose of about 5 mg of sonrotoclax per day from Day 8 to 10, followed by a dose of about 10 mg of sonrotoclax per day from Day 11 to 14, followed by a dose of about 20 mg of sonrotoclax per day from Day 15 to 17, followed by a dose of about 40 mg of sonrotoclax per day from Day 18 to 21, followed by a dose of about 80 mg of sonrotoclax per day from Day 22 to 24, followed by a dose of about 160 mg of sonrotoclax per day from Day 25 to 28, and the escalating doses are followed by a therapeutically effective target daily dose of about 320 mg of sonrotoclax starting from Day 29, wherein the patient has a B-cell malignancy, particularly CLL/SLL or MCL, more particularly R/R CLL/SLL or R/R MCL.

Provided herein is also a method for improving ORR comprising administering to patients in need thereof sonrotoclax or a pharmaceutically acceptable salt thereof in therapeutically effective escalating doses, wherein the escalating doses comprise a dose of about 1 mg of sonrotoclax per day from Day 1 to 3, followed by a dose of about 2 mg of sonrotoclax per day from Day 4 to 7, followed by a dose of about 5 mg of sonrotoclax per day from Day 8 to 10, followed by a dose of about 10 mg of sonrotoclax per day from Day 11 to 14, followed by a dose of about 20 mg of sonrotoclax per day from Day 15 to 17, followed by a dose of about 40 mg of sonrotoclax per day from Day 18 to 21, followed by a dose of about 80 mg of sonrotoclax per day from Day 22 to 24, followed by a dose of about 160 mg of sonrotoclax per day from Day 25 to 28, and the escalating doses are followed by a therapeutically effective target daily dose of about 320 mg of sonrotoclax starting from Day 29, wherein the patient has a B-cell malignancy, particularly CLL/SLL or MCL, more particularly R/R CLL/SLL or R/R MCL.

Provided herein is also a method for treating a B-cell malignancy, particularly CLL/SLL or MCL, more particularly R/R CLL, R/R SLL, or R/R MCL comprising
administering to a patient in need thereof sonrotoclax or a pharmaceutically acceptable salt thereof in therapeutically effective escalating doses followed by a therapeutically effective target daily dose;
interrupting the administration of sonrotoclax or a pharmaceutically acceptable salt thereof if the patient experiences an adverse reaction; and
restarting the administration of sonrotoclax or a pharmaceutically acceptable salt thereof after the patient is recovered,
wherein:
the adverse reaction is an event suggestive of TLS, particularly clinical TLS, a hematological toxicity or a nonhematological toxicity;
when the interruption occurs during the administration of escalating doses,
   if the dose interruption lasts up to 7 days, the administration is restarted at the same dose as planned prior to the interruption, and
   if the dose interruption lasts greater than 7 days, the administration is restarted at least one dose level lower than the dose as planned prior to the interruption except when the dose as planned prior to the interruption is the initial dose of about 1 mg, then the administration is restarted at about 1 mg; and
when the dose interruption occurs during the administration of the target daily dose,
   after the patient is recovered to baseline or Grade 1 toxicity from the first adverse reaction, if the first adverse reaction is an event suggestive of TLS, a hematological toxicity or a Grade 3 nonhematological toxicity, the administration is restarted at the target daily dose, and if the first adverse reaction is a Grade 4 nonhematological toxicity, the administration is restarted at 50% of the target daily dose,
   after the patient is recovered from the second and subsequent adverse reactions, the administration is restarted at a daily dose that is 50% of the dose prior to the interruption of the administration, and
   if the patient experiences an adverse reaction after two dose reductions, the administration of sonrotoclax or a pharmaceutically acceptable salt thereof is discontinued.

Provided herein is also a method of administering sonrotoclax or a pharmaceutically acceptable salt thereof comprising:
administering to a patient in need thereof sonrotoclax or a pharmaceutically acceptable salt thereof in therapeutically effective escalating doses,
interrupting the administration of sonrotoclax or a pharmaceutically acceptable salt thereof if the patient experiences an adverse reaction, and
restarting the administration of sonrotoclax or a pharmaceutically acceptable salt thereof after the patient is recovered,
wherein:
the adverse reaction is an event suggestive of TLS, particularly clinical TLS, a hematological toxicity or a nonhematological toxicity,
when the interruption lasts up to 7 days, the administration is restarted at the same dose as planned prior to the interruption,
when the interruption lasts greater than 7 days, the administration is restarted at least one dose level lower than the dose as planned prior to the interruption except when the dose as planned prior to the interruption is the initial dose of about 1 mg, then the administration is restarted at about 1 mg, and
sonrotoclax or a pharmaceutically acceptable salt thereof is being administered to treat a B-cell malignancy, particularly CLL/SLL or MCL, and more particularly R/R CLL/SLL or R/R MCL.

Provided herein is also a method of administering sonrotoclax or a pharmaceutically acceptable salt thereof comprising:
administering to a patient in need thereof a therapeutically effective target daily dose of sonrotoclax or a pharmaceutically acceptable salt thereof to treat a B-cell malignancy, particularly CLL/SLL or MCL, and more particularly R/R CLL/SLL or R/R MCL,
interrupting the administration of sonrotoclax or a pharmaceutically acceptable salt thereof if the patient experiences an adverse reaction, wherein the adverse reaction is an event suggestive of TLS, particularly clinical TLS, a hematological toxicity or a nonhematological toxicity, and
restarting the administration of sonrotoclax or a pharmaceutically acceptable salt thereof after the patient is recovered,
wherein:
after the patient is recovered to baseline or Grade 1 toxicity from the first adverse reaction, if the first adverse reaction is an event suggestive of TLS, a hematological toxicity or a Grade 3 nonhematological toxicity, the administration is restarted at the target daily dose, and if the first adverse reaction is a Grade 4 nonhematological toxicity, the administration is restarted at 50% of the target daily dose
after the patient is recovered from the second and subsequent adverse reactions, the administration is restarted at a daily dose that is 50% of the dose prior to the interruption of the administration, and
if the patient experiences an adverse reaction after two dose reductions, the administration of sonrotoclax or a pharmaceutically acceptable salt thereof is discontinued.

In some embodiments, the therapeutically effective escalating doses of sonrotoclax comprise a dose of about 2 mg of sonrotoclax per day of Week 1, followed by a dose of about 5 mg of sonrotoclax per day of Week 2, followed by a dose of about 20 mg of sonrotoclax per day of Week 3, and followed by a dose of about 80 mg of sonrotoclax per day of Week 4.

In some embodiments, the therapeutically effective escalating doses of sonrotoclax comprise a dose of about 2 mg of sonrotoclax per day of Week 1, followed by a dose of about 5 mg of sonrotoclax per day of Week 2, followed by a dose of about 15 mg of sonrotoclax per day of Week 3, and followed by a dose of about 60 mg of sonrotoclax per day of Week 4.

In some embodiments, the therapeutically effective escalating doses of sonrotoclax comprise a dose of about 2 mg of sonrotoclax per day of Week 1, followed by a dose of about 10 mg of sonrotoclax per day of Week 2, followed by a dose of about 60 mg of sonrotoclax per day of Week 3, and followed by a dose of about 120 mg of sonrotoclax per day of Week 4.

In some embodiments, the therapeutically effective escalating doses of sonrotoclax comprise a dose of about 2 mg of sonrotoclax per day of Week 1, followed by a dose of about 5 mg of sonrotoclax per day of Week 2, followed by a dose of about 15 mg of sonrotoclax per day of Week 3, followed by a dose of about 45 mg of sonrotoclax per day of Week 4, and followed by a dose of about 140 mg of sonrotoclax per day of Week 5.

Provided herein is also a method for treating a B-cell malignancy, particularly CLL/SLL or MCL, more particularly R/R CLL, R/R SLL, or R/R MCL comprising administering to a patient in need thereof sonrotoclax or a pharmaceutically acceptable salt thereof in therapeutically effective escalating doses followed by a therapeutically effective target daily dose, wherein the escalating doses comprise a dose of about 2 mg of sonrotoclax per day of Week 1, followed by a dose of about 5 mg of sonrotoclax per day of Week 2, followed by a dose of about 20 mg of sonrotoclax per day of Week 3, and followed by a dose of about 80 mg of sonrotoclax per day of Week 4;
a dose of about 2 mg of sonrotoclax per day of Week 1, followed by a dose of about 5 mg of sonrotoclax per day of Week 2, followed by a dose of about 15 mg of sonrotoclax per day of Week 3, and followed by a dose of about 60 mg of sonrotoclax per day of Week 4;
a dose of about 2 mg of sonrotoclax per day of Week 1, followed by a dose of about 10 mg of sonrotoclax per day of Week 2, followed by a dose of about 60 mg of sonrotoclax per day of Week 3, and followed by a dose of about 120 mg of sonrotoclax per day of Week 4; or
a dose of about 2 mg of sonrotoclax per day of Week 1, followed by a dose of about 5 mg of sonrotoclax per day of Week 2, followed by a dose of about 15 mg of sonrotoclax per day of Week 3, followed by a dose of about 45 mg of sonrotoclax per day of Week 4, and followed by a dose of about 140 mg of sonrotoclax per day of Week 5.

Provided herein is also a method for treating a B-cell malignancy, particularly CLL/SLL or MCL, more particularly R/R CLL, R/R SLL, or R/R MCL while preventing TLS or reducing the risk of TLS comprising administering to a patient in need thereof sonrotoclax or a pharmaceutically acceptable salt thereof in therapeutically effective escalating doses followed by a therapeutically effective target daily dose, wherein the escalating doses comprise
a dose of about 2 mg of sonrotoclax per day of Week 1, followed by a dose of about 5 mg of sonrotoclax per day of Week 2, followed by a dose of about 20 mg of sonrotoclax per day of Week 3, and followed by a dose of about 80 mg of sonrotoclax per day of Week 4;
a dose of about 2 mg of sonrotoclax per day of Week 1, followed by a dose of about 5 mg of sonrotoclax per day of Week 2, followed by a dose of about 15 mg of sonrotoclax per day of Week 3, and followed by a dose of about 60 mg of sonrotoclax per day of Week 4;
a dose of about 2 mg of sonrotoclax per day of Week 1, followed by a dose of about 10 mg of sonrotoclax per day of Week 2, followed by a dose of about 60 mg of sonrotoclax per day of Week 3, and followed by a dose of about 120 mg of sonrotoclax per day of Week 4; or
a dose of about 2 mg of sonrotoclax per day of Week 1, followed by a dose of about 5 mg of sonrotoclax per day of Week 2, followed by a dose of about 15 mg of sonrotoclax per day of Week 3, followed by a dose of about 45 mg of sonrotoclax per day of Week 4, and followed by a dose of about 140 mg of sonrotoclax per day of Week 5.

Provided herein is also a method for preventing TLS or reducing the risk of TLS comprising administering to a patient in need thereof sonrotoclax or a pharmaceutically acceptable salt thereof in therapeutically effective escalating doses followed by a therapeutically effective target daily dose, wherein the escalating doses comprise
a dose of about 2 mg of sonrotoclax per day of Week 1, followed by a dose of about 5 mg of sonrotoclax per day of Week 2, followed by a dose of about 20 mg of sonrotoclax per day of Week 3, and followed by a dose of about 80 mg of sonrotoclax per day of Week 4;
a dose of about 2 mg of sonrotoclax per day of Week 1, followed by a dose of about 5 mg of sonrotoclax per day of Week 2, followed by a dose of about 15 mg of sonrotoclax per day of Week 3, and followed by a dose of about 60 mg of sonrotoclax per day of Week 4;
a dose of about 2 mg of sonrotoclax per day of Week 1, followed by a dose of about 10 mg of sonrotoclax per day of Week 2, followed by a dose of about 60 mg of sonrotoclax per day of Week 3, and followed by a dose of about 120 mg of sonrotoclax per day of Week 4; or
a dose of about 2 mg of sonrotoclax per day of Week 1, followed by a dose of about 5 mg of sonrotoclax per day of Week 2, followed by a dose of about 15 mg of sonrotoclax per day of Week 3, followed by a dose of about 45 mg of sonrotoclax per day of Week 4, and followed by a dose of about 140 mg of sonrotoclax per day of Week 5.

Provided herein is also a method for increasing response rate comprising administering to a patient in need thereof sonrotoclax or a pharmaceutically acceptable salt thereof in therapeutically effective escalating doses, wherein the escalating doses comprise
a dose of about 2 mg of sonrotoclax per day of Week 1, followed by a dose of about 5 mg of sonrotoclax per day of Week 2, followed by a dose of about 20 mg of sonrotoclax per day of Week 3, and followed by a dose of about 80 mg of sonrotoclax per day of Week 4;
a dose of about 2 mg of sonrotoclax per day of Week 1, followed by a dose of about 5 mg of sonrotoclax per day of Week 2, followed by a dose of about 15 mg of sonrotoclax per day of Week 3, and followed by a dose of about 60 mg of sonrotoclax per day of Week 4;
a dose of about 2 mg of sonrotoclax per day of Week 1, followed by a dose of about 10 mg of sonrotoclax per day of Week 2, followed by a dose of about 60 mg of sonrotoclax per day of Week 3, and followed by a dose of about 120 mg of sonrotoclax per day of Week 4; or
a dose of about 2 mg of sonrotoclax per day of Week 1, followed by a dose of about 5 mg of sonrotoclax per day of Week 2, followed by a dose of about 15 mg of sonrotoclax per day of Week 3, followed by a dose of about 45 mg of sonrotoclax per day of Week 4, and followed by a dose of about 140 mg of sonrotoclax per day of Week 5.

Provided herein is also a method for improving ORR comprising administering to patients in need thereof sonrotoclax or a pharmaceutically acceptable salt thereof in therapeutically effective escalating doses, wherein the escalating doses comprise
a dose of about 2 mg of sonrotoclax per day of Week 1, followed by a dose of about 5 mg of sonrotoclax per day of Week 2, followed by a dose of about 20 mg of sonrotoclax per day of Week 3, and followed by a dose of about 80 mg of sonrotoclax per day of Week 4;
a dose of about 2 mg of sonrotoclax per day of Week 1, followed by a dose of about 5 mg of sonrotoclax per day of Week 2, followed by a dose of about 15 mg of sonrotoclax per day of Week 3, and followed by a dose of about 60 mg of sonrotoclax per day of Week 4;
a dose of about 2 mg of sonrotoclax per day of Week 1, followed by a dose of about 10 mg of sonrotoclax per day of Week 2, followed by a dose of about 60 mg of sonrotoclax per day of Week 3, and followed by a dose of about 120 mg of sonrotoclax per day of Week 4; or
a dose of about 2 mg of sonrotoclax per day of Week 1, followed by a dose of about 5 mg of sonrotoclax per day of Week 2, followed by a dose of about 15 mg of sonrotoclax per day of Week 3, followed by a dose of about 45 mg of sonrotoclax per day of Week 4, and followed by a dose of about 140 mg of sonrotoclax per day of Week 5.

In some embodiments, sonrotoclax is administered with food. In some embodiments, sonrotoclax is administered without food.

In some embodiments, the patient is an adult patient having R/R MCL after at least two prior systemic therapies, including a BTK inhibitor.

In some embodiments, the patient is an adult patient having MCL who have previously received a Bruton Tyrosine Kinase inhibitor (BTKi) therapy.

In some embodiments, the patient is an adult patient having MCL, who has received at least one line of anti-CD20-based therapy and at least one line of BTK inhibitor therapy. In some embodiments, the patient is an adult patient having MCL, who has been previously treated with a BTK inhibitor and anti-CD20 antibody.

In some embodiments, the patient is an adult patient having CLL/SLL who received at least one prior systemic therapy including a BTK inhibitor. In some embodiments, the patient is an adult patient having CLL/SLL who has previously received at least one systemic therapy including a BTK inhibitor.

In some embodiments, the patient is an adult patient who received previous treatment for CLL or SLL.

In some embodiments, the patient is an adult patient having MCL, who has received at least one line of anti-CD20-based therapy and at least one line of BTK inhibitor therapy.

In some embodiments, the BTK inhibitor is a non-covalent BTK inhibitor.

In some embodiments, the BTK inhibitor is a covalent BTK inhibitor.

In some embodiments, the B-cell malignancy is previously untreated (TN) CLL or TN SLL.

In some embodiments, sonrotoclax is ramped up to a target daily dose according to the ramp-up schedule provided here, wherein when the target daily dose is reached, the following ramp up steps are not used.

### EXAMPLES

The examples below are intended to be purely exemplary and should not be considered to be limiting in any way.

The following lists abbreviations used in the Example section:

| Abbreviation | Definition |
|---|---|
| ANC | absolute neutrophil count |
| ASCT | autologous stem cell transplant |
| BIW | Twice-a-week |
| BR | bendamustine plus rituximab |
| CI | Confidence Interval |
| CNS | central nervous system |
| CR | Complete response |
| CRi | complete response with incomplete bone marrow recovery |
| LDi | longest transverse diameter of a lesion |
| NE | Not estimable |
| NED | No evidence of disease |
| NGS | next-generation sequencing |
| nPR | nodular partial response |
| ORR | Overall response rate |
| PD | Progressive disease |
| PR | Partial response |
| PRO | patient-reported outcome |
| PT | Preferred Term |
| SAE | serious adverse event |
| SD | standard deviation or stable disease. |
| TEAE | treatment-emergent adverse event |
| W | week |

### Example 1—Clinical Study of Sonrotoclax in Patients with R/R CLL or R/R SLL

This is an ongoing single-arm, open-label, multicenter Phase 2 study to evaluate the safety and efficacy of sonrotoclax in patients with R/R CLL or R/R SLL.

Patient inclusion criteria: Patients were screened before the treatment. In addition to signed informed consent, each patient eligible to participate in this study must meet all of the following inclusion criteria: (1) age 18 years or older; and (2) patients with a histologically confirmed diagnosis of CLL/SLL based on the International Workshop on CLL (iwCLL) criteria who have: (a) treatment intolerance or failure during or after treatment with CIT and BTK inhibitors; or (b) treatment intolerance or failure during or after treatment with BTKi for CIT ineligible patients.

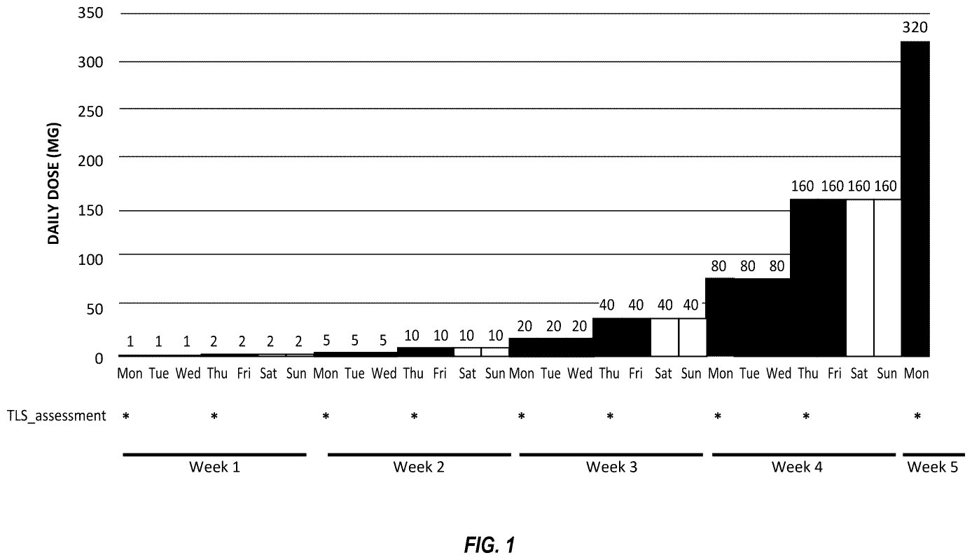

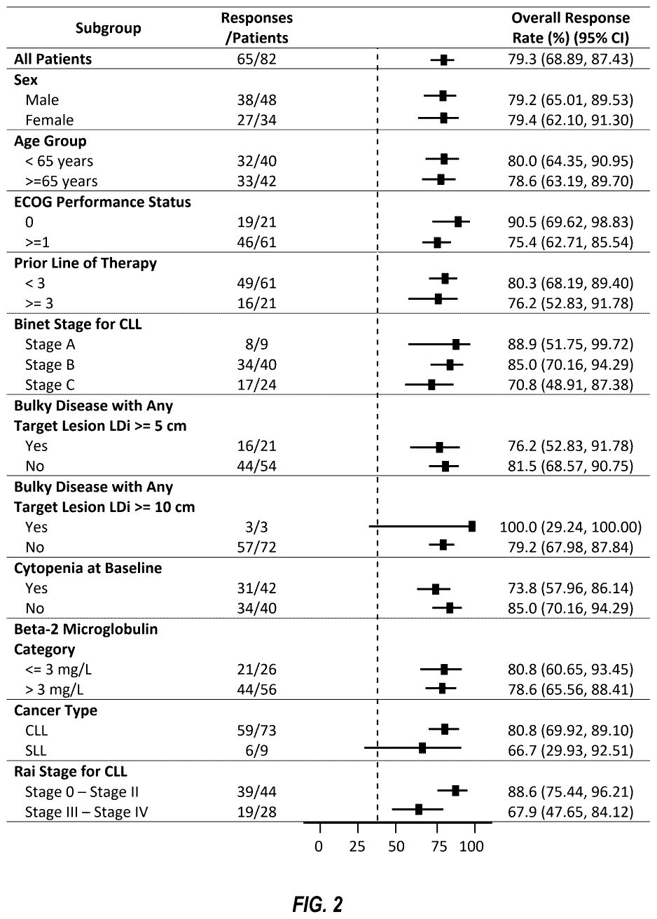

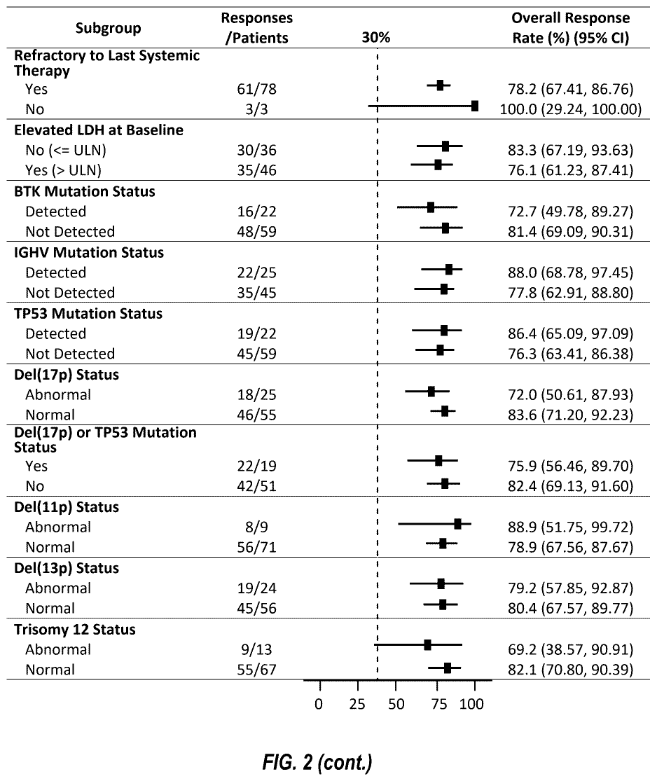

Dose and mode of administration: For all patients, antihyperuricemic treatment (allopurinol, febuxostat, or other uric acid reducers per institutional standards) were given 2 to 3 days prior to the first dose and continue until 3 to 7 days after reaching the target daily dose. Sonrotoclax was given to eligible patients at 320 mg once daily starting with (1) a 5-week twice-a-week ramp-up schedule with gradual incremental of doses (1 mg, 2 mg, 5 mg, 10 mg, 20 mg, 40 mg, 80 mg, 160 mg, and 320 mg) as depicted in Table 1 and FIG. 1, followed by 320 mg of sonrotoclax once per day, or (2) a 9-week once-a-week ramp-up schedule with gradual incremental of doses (1 mg, 2 mg, 5 mg, 10 mg, 20 mg, 40 mg, 80 mg, 160 mg, and 320 mg) as depicted in Table 2, followed by 320 mg of sonrotoclax once per day.

TABLE 1
Dosing Schedule for 5-Week Twice-a-week Ramp-up Phase

| Week | Days | Daily Dose |
|---|---|---|
| Week 1 | Days 1 to 3 | 1 mg |
| | Days 4 to 7 | 2 mg |
| Week 2 | Days 8 to 10 | 5 mg |
| | Days 11 to 14 | 10 mg |
| Week 3 | Days 15 to 17 | 20 mg |
| | Days 18 to 21 | 40 mg |
| Week 4 | Days 22 to 24 | 80 mg |
| | Days 25 to 28 | 160 mg |

| | |
|---|---|
| Week 5 and beyond | 320 mg |

TABLE 2
Dosing Schedule for 9-Week Once-a-week Ramp-up Phase

| Week number | Dose (mg once per day) |
|---|---|
| 1 | 1 |
| 2 | 2 |
| 3 | 5 |
| 4 | 10 |
| 5 | 20 |
| 6 | 40 |
| 7 | 80 |
| 8 | 160 |
| 9 and beyond | 320 |

Duration of the treatment: Patients received sonrotoclax until disease progression, unacceptable toxicity, withdrawal of consent, death, or loss to follow-up, investigator's decision, or study termination by the sponsor, whichever occurs first.

Criteria for evaluation: The primary efficacy endpoint was the ORR determined by Independent Review Committee (IRC).

The main secondary efficacy endpoints were: (1) ORR as determined by the investigator; (2) DoR as determined by the IRC and the investigator, defined as the time from first determination of response until first documentation of progression or death, whichever occurs first; (3) PFS as determined by the IRC and the investigator, defined as the time from start of treatment to the first documentation of disease progression or death, whichever occurs first; (4) TTR as determined by the IRC and the investigator, defined as the time from start of treatment to first documentation of response; and (5) OS as defined as the time from the start of treatment to the date of death due to any cause.

The safety analyses were conducted based on the Safety Analysis Set. Descriptive statistics were used to analyze all safety data in the Safety Analysis Set.

Methodology: Response was assessed both by the IRC and the investigators, based on disease-related constitutional symptoms, physical examinations, laboratory results, computed tomography (CT) or magnetic resonance imaging (MRI), and bone marrow evaluation according to International Workshop on CLL (iwCLL) Guidelines in patients with CLL and to the Lugano Classification for non-Hodgkin lymphoma in patients with SLL. Response assessments started from Cycle 3 Day 1 and continued every 3 cycles through Cycle 12 and then every 6 cycles thereafter or when a significant change in response is suspected (progressive disease or upgrade in response). Clinical suspicion of disease progression at any time required that physical examination and hematological and radiologic confirmation to be performed promptly, rather than waiting for the next scheduled radiological assessment. Patients should remain on study treatment until disease progression was confirmed by the IRC. Patients who met the clinical and laboratory criteria for complete response (CR) or complete response with incomplete bone marrow recovery (CRi) needed bone marrow examination to confirm CR or CRi. This was collected within 28 days after the CT/MRI assessment meeting the criteria for CR or CRi. All other clinical data were collected within +14 days after the CT/MRI (i.e., complete blood count with differential and physical examination).

Safety assessments included adverse events (AEs), serious adverse events (SAEs), clinical laboratory tests, physical examinations, electrocardiograms, and vital signs. AEs were graded for severity per the National Cancer Institute Common Terminology Criteria for Adverse Events version 5.0 (NCI-CTCAE v5.0) or the Grading Scale for Hematologic Toxicities in CLL Studies as appropriate.

A Safety Monitoring Committee periodically monitored the safety data from all patients.

### Dose Interruption and Modification

Dosing was held for individual patients for clinically relevant TLS-related laboratory abnormalities, TLS (laboratory or clinical), hematologic toxicities and nonhematologic toxicities, based on investigator assessment of study drug relatedness:

### Hematologic Toxicities

Grade 3 or higher febrile neutropenia
Grade 3 or higher thrombocytopenia that results in clinically significant bleeding
Any ≥Grade 4 hematological toxicity with the following exceptions:
   Grade 4 neutropenia lasting ≤7 days with or without treatment
   Grade 4 lymphopenia
   Grade 4 leukopenia

Any ≥Grade 3 nonhematologic toxicity with the following exceptions:
TLS-related lab AEs (ie, hyperuricemia, hyperphosphatemia, hyperkalemia, and/or hypocalcemia) that resolve ≤3 days with or without treatment
Grade 3 gastrointestinal toxicity (ie, nausea, vomiting, diarrhea) unless unresponsive to treatment ≥7 days
Asymptomatic biochemical lab abnormalities that resolve ≤7 days

Sonrotoclax may be restarted after resolution of the toxicity. If the dose interruption occurred during the ramp-up and lasted ≤7 days, sonrotoclax may be restarted at the same dose level as before the interruption or at 1 dose level lower, per investigator's discretion based on a risk assessment (including tumor burden status). If the dose interruption occurred during the ramp-up and lasted >7 days, the restarting dose must be ≥1 dose level lower than the last dose given prior to the interruption (Table 3), except for the initial dose of 1 mg.

TABLE 3
Dose to Restart Sonrotoclax After a Dose Interruption >7 Days

| Dose at time of interruption (mg) | Highest restart dose (mg)* |
|---|---|
| 1 mg | 1 mg |
| 2 mg | 1 mg |
| 5 mg | 2 mg |
| 10 mg | 5 mg |
| 20 mg | 10 mg |
| 40 mg | 20 mg |
| 80 mg | 40 mg |
| 160 mg | 80 mg |
| 320 mg | 160 mg |

*The investigator may restart at a lower dose than noted above.

If dose interruption occurred during the daily dose of 320 mg administration, sonrotoclax may be restarted according to Table 4 and a maximum of 2 dose reductions were allowed.

TABLE 4
Sonrotoclax Dose Reduction Levels

| | | Sonrotoclax dose upon |
|---|---|---|
| Toxicity occurrence | Dose level | resumption of dosing |
| First | 320 mg QD | 320 mg QD |
| Second | 320 mg QD | 160 mg QD |
| Third | 160 mg QD | 80 mg QD |
| Fourth | 80 mg QD | Discontinue sonrotoclax |

### Results as of the First Data Cut Off Date

Safety: As of the first data cut off (DCO) date, 52 patients with R/R CLL/SLL (45 with CLL and 7 with SLL) were treated with the 5-week twice-a-week (BIW) ramp-up schedule followed by 320 mg of sonrotoclax once per day. The median age of the patients was 64 years (range: 37 to 82), and 53.8% were male. The median number of prior therapies was 2 (range: 1 to 6). The median duration of treatment with sonrotoclax at the time of data analysis was 9.9 months (range: 0 to 16.4 months), 77.0% were exposed for at least 6 months, and 46.2% were exposed for more than 12 months.

Safety results for these 52 patients with R/R CLL/SLL: TEAEs were reported in 96.2% of patients, and the most frequently reported events (in ≥30% of patients) were neutrophil count decreased (40.4%), and platelet count decreased (36.5%). 51.9% of patients reported any TEAE of ≥Grade 3, and the most frequently reported events (in ≥10% of patients) were neutrophil count decreased (23.1%) and pneumonia (11.5%). 28.8% of patients reported any SAE. SAEs reported in more than 1 patient were anaemia (3.0%), death (1.9%), and pleural effusion (1.9%). 5.8% of patients discontinued from treatment due to TEAEs. 5.8% of patients had dose reduction due to TEAEs. All TEAEs leading to dose modification involved dose interruption (32.7%). The most frequently reported TEAEs that led to dose interruption involved the SOC Infections and infestations (9.6%).

As of the first DCO date, 48 patients with R/R CLL/SLL (45 with CLL and 3 with SLL) were treated with the 9-week once-a-week ramp-up schedule. The median age of the patients was 66 years (range: 40 to 78), and 68.8% were male. The median number of prior therapies was 2 (range: 1 to 6). The median duration of treatment with sonrotoclax at the time of data analysis was 17.1 months (range: 0.6 to 27.5 months), 77.1% were exposed for at least 6 months, and 62.5% were exposed for more than 12 months.

Safety results for these 48 patients: TEAEs were reported in 97.9% of patients, and the most frequently reported events (in ≥30% of patients) were neutrophil count decreased (68.8%), platelet count decreased (45.8%), anaemia (43.8%), hyperuricaemia (37.5%), and white blood cell count decreased (33.3%). 70.8% of patients reported any TEAE of ≥Grade 3, and the most frequently reported events (in ≥10% of patients) were neutrophil count decreased (43.8%), pneumonia (22.9%), platelet count decreased (12.5%), and white blood cell count decreased (14.6%). 47.9% of patients reported any SAE. SAEs reported in more than 1 patient were pneumonia (22.9%), anaemia (6.3%), death (2.1%), hyperkalaemia (4.2%), pleural effusion (2.1%), and pneumonitis (4.2%). 6.3% of patients discontinued from treatment due to TEAEs. 4.2% of patients had dose reduction due to TEAEs. All TEAEs leading to dose modification involved dose interruption (45.8%). The most frequently reported TEAEs that led to dose interruption involved the SOC Infections and infestations (25.0%).

TLS: As of the first DCO date, for the 52 patients treated with the 5W BIW ramp-up schedule, no clinical TLS were observed. 4 laboratory TLS were reported by the instigators and were all resolved. For the 48 patients treated with the weekly ramp-up schedule, at the beginning of the enrollment, the TLS monitoring procedure was not fully established. Once the TLS monitoring was established, no events of laboratory or clinical TLS were reported by investigators. In addition, an algorithmic search was performed using the Howard Criteria to identify cases of laboratory TLS based on postdose laboratory data within 7 days of sonrotoclax initiation or planned dose increase. Eight patients (8.0% of total patients) had ≥2 postdose laboratory abnormalities indicative of laboratory TLS, in which 3 patients followed the weekly ramp-up schedule (6.3% of weekly ramp-up schedule population) and 5 patients followed the BIW ramp-up schedule (9.6% of BIW ramp-up schedule population).

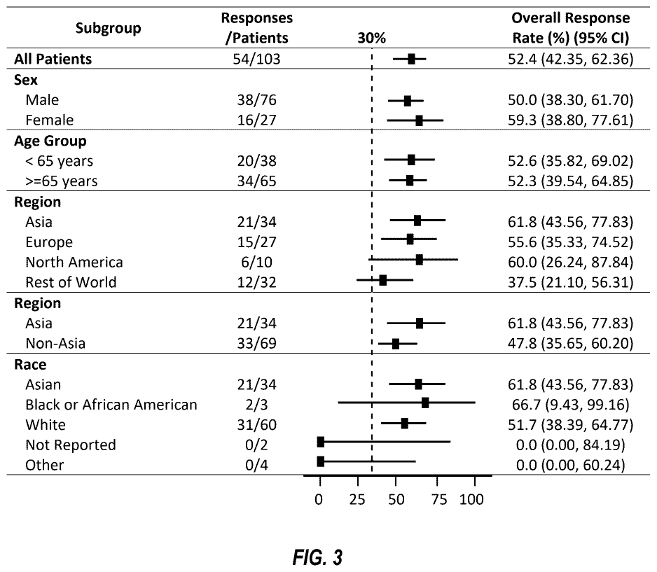

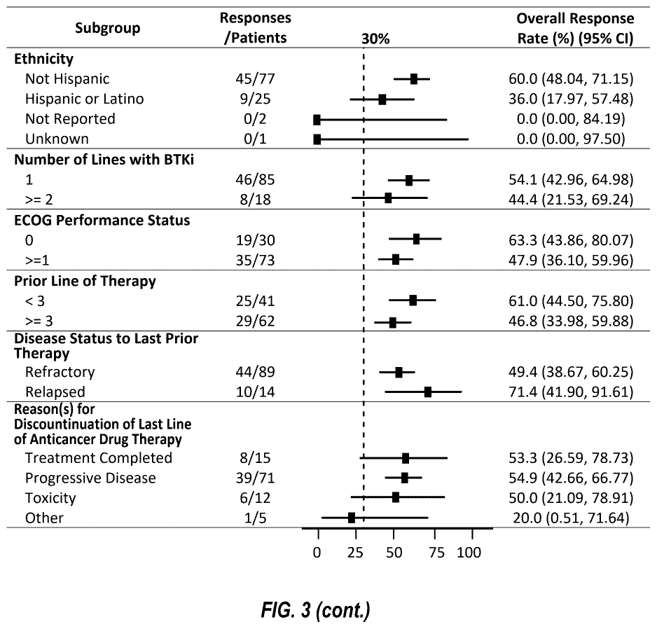

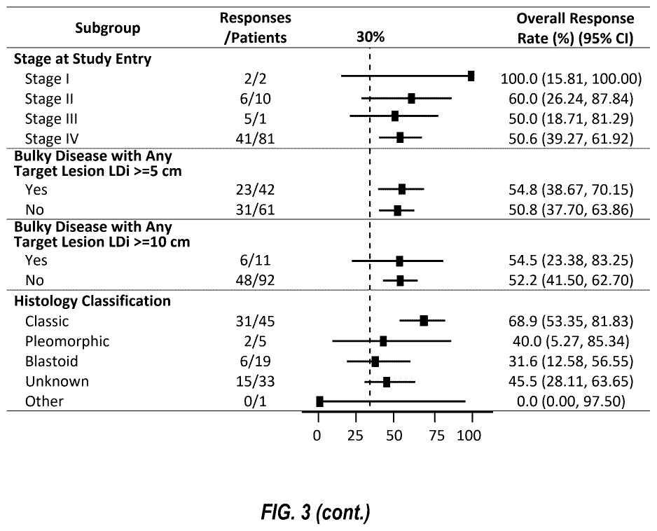

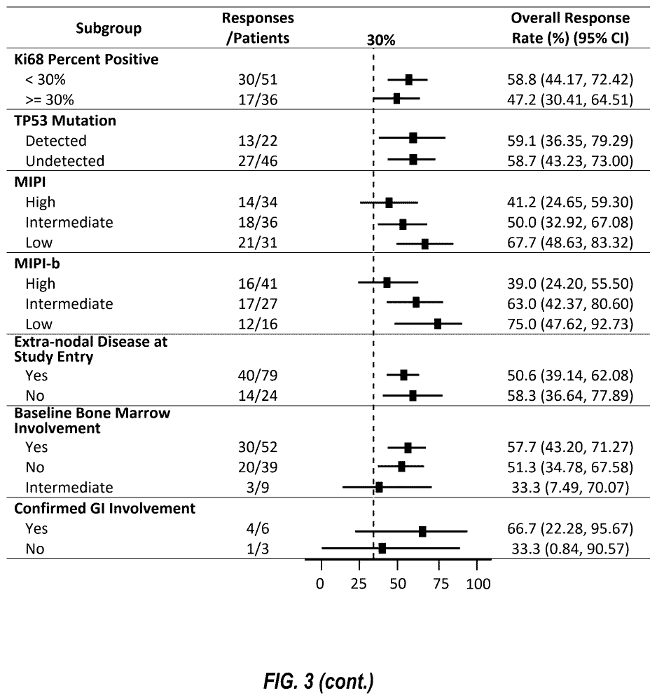

Efficacy: The efficacy results are shown in Table 5-Table 9 and FIG. 3. DOR, PFS, and OS were not mature as of the first DCO date, with median DOR, PFS, and OS not having been reached.

TABLE 5
Analysis of Disease Response by IRC

| | Sonroto- | Sonroto- | Sonroto- | Sonroto- |
|---|---|---|---|---|
| | clax | clax | clax | clax |
| | Weekly | BIW | Total | Total * |
| Response Category | (N = 48) | (N = 52) | (N = 100) | (N = 82) |
| Best Overall Response, | | | | |
| n (%) | | | | |
| CR | 9 (18.8) | 8 (15.4) | 17 (17.0) | 15 (18.3) |
| CRi | 1 (2.1) | 1 (1.9) | 2 (2.0) | 2 (2.4) |
| nPR | 0 (0.0) | 0 (0.0) | 0 (0.0) | 0 (0.0) |
| PR | 24 (50.0) | 33 (63.5) | 57 (57.0) | 48 (58.5) |
| SD | 5 (10.4) | 4 (7.7) | 9 (9.0) | 6 (7.3) |
| PD | 4 (8.3) | 3 (5.8) | 7 (7.0) | 5 (6.1) |
| Could Not Be Determined | 5 (10.4) | 3 (5.8) | 8 (8.0) | 6 (7.3) |
| Not Evaluable (NED or | 1 (2.1) | 0 (0.0) | 1 (1.0) | 1 (1.2) |
| Unknown or non-PD) | | | | |
| Not Assessable ^{a} | 4 (8.3) | 3 (5.8) | 7 (7.0) | 5 (6.1) |
| Overall Response Rate, | 34 (70.8) | 42 (80.8) | 76 (76.0) | 65 (79.3) |
| n (%) ^{b} | | | | |
| 95% CI (%) ^{c} | 55.9, 83.0 | 67.5, 90.4 | 66.4, 84.0 | 68.9, 87.4 |
| 1-Sided p-Value ^{d} | — | — | — | <0.0001 |
| Complete Response Rate, | 10 (20.8) | 9 (17.3) | 19 (19.0) | 17 (20.7) |
| n (%) ^{e} | | | | |
| 95% CI (%) ^{c} | 10.5, 35.0 | 8.2, 30.3 | 11.8, 28.1 | 12.6, 31.1 |

TABLE 6
Analysis of Disease Response by Investigator

| | Sonroto- | Sonroto- | Sonroto- | Sonroto- |
|---|---|---|---|---|
| | clax | clax | clax | clax |
| | Weekly | BIW | Total | Total * |
| Response Category | (N = 48) | (N = 52) | (N = 100) | (N = 82) |
| Best Overall Response, | | | | |
| n (%) | | | | |
| CR | 11 (22.9) | 7 (13.5) | 18 (18.0) | 13 (15.9) |
| CRi | 0 (0.0) | 0 (0.0) | 0 (0.0) | 0 (0.0) |
| nPR | 0 (0.0) | 0 (0.0) | 0 (0.0) | 0 (0.0) |
| PR | 22 (45.8) | 32 (61.5) | 54 (54.0) | 48 (58.5) |
| SD | 5 (10.4) | 7 (13.5) | 12 (12.0) | 10 (12.2) |
| PD | 6 (12.5) | 3 (5.8) | 9 (9.0) | 6 (7.3) |
| Could Not Be Determined | 4 (8.3) | 3 (5.8) | 7 (7.0) | 5 (6.1) |
| Possible Tumor Flare | 0 (0.0) | 0 (0.0) | 0 (0.0) | 0 (0.0) |
| Not Evaluable | 0 (0.0) | 0 (0.0) | 0 (0.0) | 0 (0.0) |
| Not Assessable ^{a} | 4 (8.3) | 3 (5.8) | 7 (7.0) | 5 (6.1) |
| Overall Response Rate, | 33 (68.8) | 39 (75.0) | 72 (72.0) | 61 (74.4) |
| n (%) ^{b} | | | | |
| 95% CI (%) ^{c} | 53.7, 81.3 | 61.1, 86.0 | 62.1, 80.5 | 63.6, 83.4 |
| Complete Response Rate, | 11 (22.9) | 7 (13.5) | 18 (18.0) | 13 (15.9) |
| n (%) ^{d} | | | | |
| 95% CI (%) ^{c} | 12.0, 37.3 | 5.6, 25.8 | 11.0, 26.9 | 8.7, 25.6 |

TABLE 7
Time to Response by IRC

| | Sonroto- | Sonroto- | Sonroto- | Sonroto- |
|---|---|---|---|---|
| | clax | clax | clax | clax |
| | Weekly | BIW | Total | Total * |
| | (N = 48) | (N = 52) | (N = 100) | (N = 82) |
| Time to Response | | | | |
| (Months) | | | | |
| n | 34 | 42 | 76 | 65 |
| Mean (SD) | 4.23 (1.330) | 3.66 (1.644) | 3.92 (1.528) | 3.91 (1.635) |
| Median | 3.81 | 2.83 | 3.70 | 3.68 |
| Q1, Q3 | 3.71, 4.27 | 2.79, 4.27 | 2.83, 4.27 | 2.79, 4.27 |
| Min, Max | 1.3, 9.3 | 2.7, 11.1 | 1.3, 11.1 | 1.3, 11.1 |

TABLE 8
Time to Response by Investigator

| | Sonroto- | Sonroto- | Sonroto- | Sonroto- |
|---|---|---|---|---|
| | clax | clax | clax | clax |
| | Weekly | BIW | Total | Total * |
| | (N = 48) | (N = 52) | (N = 100) | (N = 82) |
| Time to Response | | | | |
| (Months) | | | | |
| n | 33 | 39 | 72 | 61 |
| Mean (SD) | 4.59 (1.444) | 3.60 (1.742) | 4.05 (1.676) | 3.99 (1.744) |
| Median | 3.88 | 2.83 | 3.70 | 3.65 |
| Q1, Q3 | 3.71, 5.09 | 2.79, 3.61 | 2.83, 4.27 | 2.79, 4.17 |
| Min, Max | 3.1, 9.3 | 2.7, 11.0 | 2.7, 11.0 | 2.7, 11.0 |

TABLE 9
Analysis of MRD Response

| | Sonroto- | Sonroto- | Sonroto- | Sonroto- |
|---|---|---|---|---|
| | clax | clax | clax | clax |
| | Weekly | BIW | Total | Total * |
| | (N = 48) | (N = 52) | (N = 100) | (N = 82) |
| Best Blood MRD Status, | | | | |
| n (%) | | | | |
| <10\hat{ }-4 | 21 (43.8) | 28 (53.8) | 49 (49.0) | 41 (50.0) |
| >=10\hat{ }-4 | 9 (18.8) | 9 (17.3) | 18 (18.0) | 15 (18.3) |
| Not Evaluable | 5 (10.4) | 2 (3.8) | 7 (7.0) | 5 (6.1) |
| MRD <10\hat{ }-4 but Total | 0 (0.0) | 0 (0.0) | 0 (0.0) | 0 (0.0) |
| Nucleated Cell Count | | | | |
| <200,000 | | | | |
| Non-Typical CLL Phenotype | 0 (0.0) | 0 (0.0) | 0 (0.0) | 0 (0.0) |
| Baseline MRD <10\hat{ }-4 | 2 (4.2) | 2 (3.8) | 4 (4.0) | 4 (4.9) |
| No Baseline | 3 (6.3) | 0 (0.0) | 3 (3.0) | 1 (1.2) |
| Missing | 13 (27.1) | 13 (25.0) | 26 (26.0) | 21 (25.6) |
| Missing Due to | 13 (27.1) | 11 (21.2) | 24 (24.0) | 19 (23.2) |
| PD/Death/Withdrawal From | | | | |
| Study/Other EOT Reason | | | | |
| Reach MRD Assessment | 0 (0.0) | 1 (1.9) | 1 (1.0) | 1 (1.2) |
| Timepoint but Missing | | | | |
| On-treatment but not Reach | 0 (0.0) | 1 (1.9) | 1 (1.0) | 1 (1.2) |
| MRD Assessment Timepoint | | | | |

The first 18 patients enrolled are excluded for registration purpose per the requirement of Center of Drug Evaluation (CDE) in China
^{a} Patients didn't have a post-baseline assessment.
^{b} b Overall response is defined as achieving a best overall response of CR, CRi, nPR, or PR for CLL and CR or PR for SLL.
^{c} The 95% CI was estimated using the Clopper-Pearson method.
^{d} The p-value was calculated from the binomial exact test of Sonrotoclax versus historical rate of 0.4.
^{e} Complete response is defined as achieving a best overall response of CR and CRi for CLL and CR for SLL.

The benefit of sonrotoclax monotherapy as assessed by ORR per IRC was observed across all analyzed subgroups as depicted in FIG. 3. In FIG. 3, the first 18 patients enrolled on or before May 6, 2023, were excluded. Mutational status of TP53 and BTK assessed by next generation sequencing with 1% sensitivity. The threshold of reporting TP53, and BTK mutation was a variant allele frequency of >5% and ≥1%, respectively.

### Results as of the Second DCO Date

Safety: As of the second DCO date, for the 52 patients with R/R CLL/SLL (45 with CLL and 7 with SLL) treated with the 5-week twice-a-week (BIW) ramp-up schedule followed by 320 mg of sonrotoclax once per day, the median duration of treatment with sonrotoclax at the time of data analysis was 13.7 months (range: 0 to 22.4 months). 76.9% of the 52 patients were exposed for at least 6 months, and 67.3% were exposed for more than 12 months.

Safety results for these 52 patients with R/R CLL/SLL: TEAEs were reported in 96.2% of patients. The most frequently reported events (in ≥30% of patients) were neutrophil count decreased (46.2%), and platelet count decreased (38.5%). 57.7% of patients reported any TEAE of ≥Grade 3, and the most frequently reported events (in ≥10% of patients) were neutrophil count decreased (23.1%) and pneumonia (11.5%). 30.8% of patients reported any SAE. SAEs reported in more than 1 patient were pneumonia (9.6%), bronchitis (1.9%), pleural effusion (1.9%), pulmonary embolism (1.9%) and death (1.9%). 5.8% of patients discontinued from treatment due to TEAEs. 5.8% of patients had dose reduction due to TEAEs. All TEAEs leading to dose modification involved dose interruption (36.5%). The most frequently reported TEAEs that led to dose interruption involved the SOC Infections and infestations (9.6%).

As of the second DCO date, for the 48 patients with R/R CLL/SLL (45 with CLL and 3 with SLL) treated with the 9-week once-a-week ramp-up schedule, the median duration of treatment with sonrotoclax at the time of data analysis was 21.0 months (range: 0.6 to 33.4 months), 77.1% were exposed for at least 6 months, and 62.5% were exposed for more than 12 months.

Safety results for these 48 patients: TEAEs were reported in 100.0% of patients, and the most frequently reported events (in ≥30% of patients) were neutrophil count decreased (70.8%), platelet count decreased (52.1%), anaemia (47.9%), hyperuricaemia (39.6%), and white blood cell count decreased (33.3%). 75.0% of patients reported any TEAE of ≥Grade 3, and the most frequently reported events (in ≥10% of patients) were neutrophil count decreased (43.8%), pneumonia (25.0%), platelet count decreased (10.4%), and white blood cell count decreased (16.7%). 52.1% of patients reported any SAE. SAEs reported in more than 1 patient were pneumonia (25.0%), anaemia (6.3%), death (2.1%), hyperkalaemia (4.2%), pleural effusion (2.1%), and pneumonitis (4.2%). 8.3% of patients discontinued from treatment due to TEAEs. 4.2% of patients had dose reduction due to TEAEs. All TEAEs leading to dose modification involved dose interruption (50.0%). The most frequently reported TEAEs that led to dose interruption involved the SOC Infections and infestations (29.2%).

TLS: As of the second DCO date, for the 52 patients treated with the 5W BIW ramp-up schedule, no clinical TLS were observed. 4 laboratory TLS were reported by the instigators and were all resolved. For the 48 patients treated with the weekly ramp-up schedule, at the beginning of the enrollment, the TLS monitoring procedure was not fully established. Once the TLS monitoring was established, no events of laboratory or clinical TLS were reported by investigators. In addition, an algorithmic search was performed using the Howard Criteria to identify cases of laboratory TLS based on postdose laboratory data within 7 days of sonrotoclax initiation or planned dose increase. Eight patients (8.0% of total patients) had ≥2 postdose laboratory abnormalities indicative of laboratory TLS, in which 3 patients followed the weekly ramp-up schedule (6.3% of weekly ramp-up schedule population) and 5 patients followed the BIW ramp-up schedule (9.6% of BIW ramp-up schedule population).

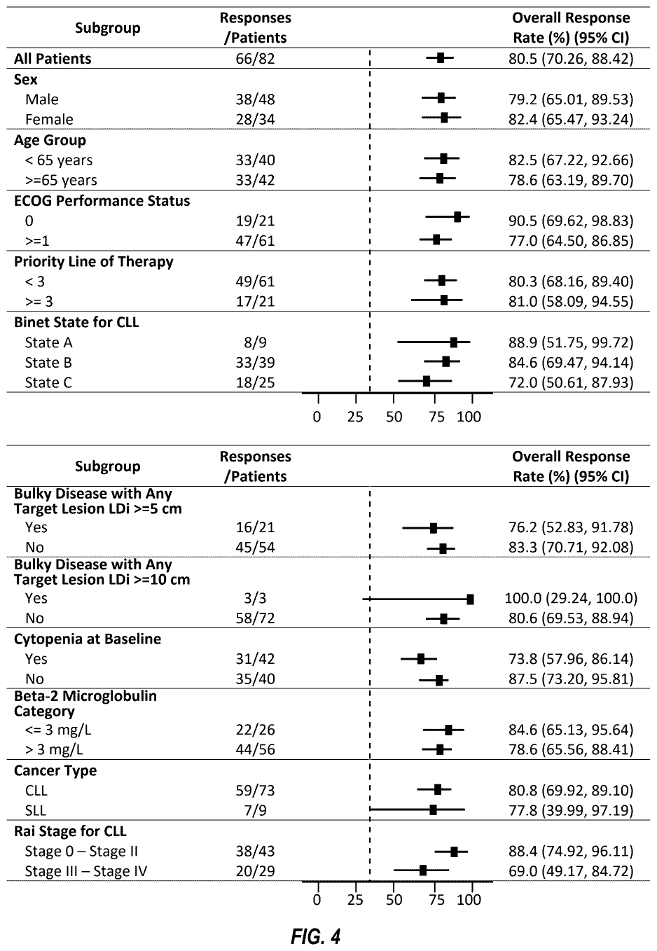

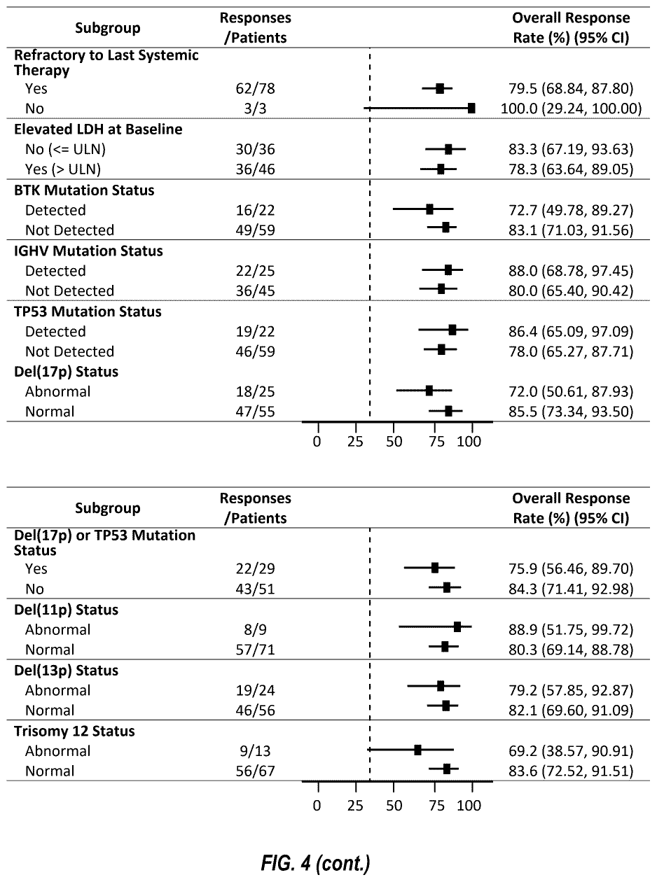

Efficacy: The efficacy results are shown in Table 10-Table 14 and FIG. 4. DOR, PFS, and OS were not mature as of the second DCO date, with median DOR, PFS, and OS not having been reached.

TABLE 10
Analysis of Disease Response by IRC

| | Sonrotoclax | Sonrotoclax | Sonrotoclax | Sonrotoclax |
|---|---|---|---|---|
| | Weekly | BIW | Total | Total * |
| Response Category | (N = 48) | (N = 52) | (N = 100) | (N = 82) |
| Best Overall Response, n (%) | | | | |
| CR | 12 (25.0) | 12 (23.1) | 24 (24.0) | 21 (25.6) |
| CRi | 1 (2.1) | 0 (0.0) | 1 (1.0) | 1 (1.2) |
| nPR | 0 (0.0) | 1 (1.9) | 1 (1.0) | 1 (1.2) |
| PR | 21 (43.8) | 30 (57.7) | 51 (51.0) | 43 (52.4) |
| SD | 5 (10.4) | 3 (5.8) | 8 (8.0) | 5 (6.1) |
| PD | 4 (8.3) | 3 (5.8) | 7 (7.0) | 5 (6.1) |
| Could Not Be Determined | 5 (10.4) | 3 (5.8) | 8 (8.0) | 6 (7.3) |
| Not Evaluable (NED or | 1 (2.1) | 0 (0.0) | 1 (1.0) | 1 (1.2) |
| Unknown or non-PD) | | | | |
| Not Assessable ª | 4 (8.3) | 3 (5.8) | 7 (7.0) | 5 (6.1) |
| Overall Response Rate, n (%) ^{b} | 34 (70.8) | 43 (82.7) | 77 (77.0) | 66 (80.5) |
| 95% CI (%) ^{c} | 55.9, 83.0 | 69.7, 91.8 | 67.5, 84.8 | 70.3, 88.4 |
| Complete Response Rate, n (%) ^{e} | 13 (27.1) | 12 (23.1) | 25 (25.0) | 22 (26.8) |
| 95% CI (%) ^{c} | 15.3, 41.8 | 12.5, 36.8 | 16.9, 34.7 | 17.6, 37.8 |

TABLE 11
Analysis of Disease Response by Investigator

| | Sonrotoclax | Sonrotoclax | Sonrotoclax | Sonrotoclax |
|---|---|---|---|---|
| | Weekly | BIW | Total | Total * |
| Response Category | (N = 48) | (N = 52) | (N = 100) | (N = 82) |
| Best Overall Response, n (%) | | | | |
| CR | 11 (22.9) | 9 (17.3) | 20 (20.0) | 16 (19.5) |
| CRi | 0 (0.0) | 0 (0.0) | 0 (0.0) | 0 (0.0) |
| nPR | 0 (0.0) | 0 (0.0) | 0 (0.0) | 0 (0.0) |
| PR | 21 (43.8) | 32 (61.5) | 53 (53.0) | 46 (56.1) |
| SD | 6 (12.5) | 5 (9.6) | 11 (11.0) | 9 (11.0) |
| PD | 6 (12.5) | 3 (5.8) | 9 (9.0) | 6 (7.3) |
| Could Not Be Determined | 4 (8.3) | 3 (5.8) | 7 (7.0) | 5 (6.1) |
| Possible Tumor Flare | 0 (0.0) | 0 (0.0) | 0 (0.0) | 0 (0.0) |
| Not Evaluable | 0 (0.0) | 0 (0.0) | 0 (0.0) | 0 (0.0) |
| Not Assessable ª | 4 (8.3) | 3 (5.8) | 7 (7.0) | 5 (6.1) |
| Overall Response Rate, n (%) ^{b} | 32 (66.7) | 41 (78.8) | 73 (73.0) | 62 (75.6) |
| 95% CI (%) ^{c} | 51.6, 79.6 | 65.3, 88.9 | 63.2, 81.4 | 64.9, 84.4 |
| Complete Response Rate, n (%) ^{d} | 11 (22.9) | 9 (17.3) | 20 (20.0) | 16 (19.5) |
| 95% CI (%) ^{c} | 12.0, 37.3 | 8.2, 30.3 | 12.7, 29.2 | 11.6, 29.7 |

TABLE 12
Time to Response by IRC

| Time to | Sonrotoclax | Sonrotoclax | Sonrotoclax | Sonrotoclax |
|---|---|---|---|---|
| Response | Weekly | BIW | Total | Total * |
| (Months) | (N = 48) | (N = 52) | (N = 100) | (N = 82) |
| n | 34 | 43 | 77 | 66 |
| Mean (SD) | 4.23 (1.330) | 3.77 (1.772) | 3.97 (1.599) | 3.97 (1.711) |
| Median | 3.81 | 2.83 | 3.71 | 3.68 |
| Q1, Q3 | 3.71, 4.27 | 2.79, 5.36 | 2.83, 4.27 | 2.79, 4.27 |
| Min, Max | 1.3, 9.3 | 2.7, 11.1 | 1.3, 11.1 | 1.3, 11.1 |

TABLE 13
Time to Response by Investigator

| Time to | Sonrotoclax | Sonrotoclax | Sonrotoclax | Sonrotoclax |
|---|---|---|---|---|
| Response | Weekly | BIW | Total | Total * |
| (Months) | (N = 48) | (N = 52) | (N = 100) | (N = 82) |
| n | 32 | 41 | 73 | 62 |
| Mean (SD) | 4.44 (1.380) | 3.70 (1.861) | 4.02 (1.697) | 3.99 (1.794) |
| Median | 3.81 | 2.83 | 3.68 | 3.63 |
| Q1, Q3 | 3.71, 4.42 | 2.79, 3.61 | 2.83, 4.17 | 2.79, 4.17 |
| Min, Max | 3.1, 9.3 | 2.7, 11.0 | 2.7, 11.0 | 2.7, 11.0 |

TABLE 14
Analysis of MRD Response

| | Sonrotoclax | Sonrotoclax | Sonrotoclax | Sonrotoclax |
|---|---|---|---|---|
| | Weekly | BIW | Total | Total * |
| | (N = 48) | (N = 52) | (N = 100) | (N = 82) |
| Best Blood MRD Status, n (%) | | | | |
| <10\hat{ }-4 | 21 (43.8) | 32 (61.5) | 53 (53.0) | 45 (54.9) |
| >=10\hat{ }-4 | 9 (18.8) | 6 (11.5) | 15 (15.0) | 12 (14.6) |
| Not Evaluable | 5 (10.4) | 2 (3.8) | 7 (7.0) | 5 (6.1) |
| MRD <10\hat{ }-4 but Total | 0 (0.0) | 0 (0.0) | 0 (0.0) | 0 (0.0) |
| Nucleated Cell Count | | | | |
| <200,000 | | | | |
| Non-Typical CLL Phenotype | 0 (0.0) | 0 (0.0) | 0 (0.0) | 0 (0.0) |
| Baseline MRD <10\hat{ }-4 | 2 (4.2) | 2 (3.8) | 4 (4.0) | 4 (4.9) |
| No Baseline | 3 (6.3) | 0 (0.0) | 3 (3.0) | 1 (1.2) |
| Missing | 13 (27.1) | 12 (23.1) | 25 (25.0) | 20 (24.4) |
| Missing Due to | 13 (27.1) | 11 (21.2) | 24 (24.0) | 19 (23.2) |
| PD/Death/Withdrawal From | | | | |
| Study/Other EOT Reason | | | | |
| Reach MRD Assessment | 0 (0.0) | 1 (1.9) | 1 (1.0) | 1 (1.2) |
| Timepoint but Missing | | | | |
| On-treatment but not Reach | 0 (0.0) | 0 (0.0) | 0 (0.0) | 0 (0.0) |
| MRD Assessment Timepoint | | | | |

The first 18 patients enrolled are excluded for registration purpose per the requirement of Center of Drug Evaluation (CDE) in China
^{a} Patients didn't have a post-baseline assessment.
^{b} Overall response is defined as achieving a best overall response of CR, CRi, nPR, or PR for CLL and CR or PR for SLL.
^{c} The 95% CI was estimated using the Clopper-Pearson method.
^{d} The p-value was calculated from the binomial exact test of Sonrotoclax versus historical rate of 0.4.
^{e} Complete response is defined as achieving a best overall response of CR and CRi for CLL and CR for SLL.

The benefit of sonrotoclax monotherapy as assessed by ORR per IRC was observed across all analyzed subgroups as depicted in FIG. 4. In FIG. 4, the first 18 patients enrolled on or before May 6, 2023, were excluded. Mutational status of TP53 and BTK assessed by next generation sequencing with 1% sensitivity. The threshold of reporting TP53, and BTK mutation was a variant allele frequency of >5% and ≥1%, respectively.

### Example 2—Clinical Study of Sonrotoclax in Patients with R/R MCL

This is an ongoing single-arm, open-label, multicenter Phase ½ study to evaluate the safety and efficacy of sonrotoclax in patients with R/R MCL.

Patient inclusion criteria: Patients were screened before the treatment. In addition to signed informed consent, each patient eligible to participate in this study must meet all of the following inclusion criteria: (1) age 18 years or older; (2) a histologically local lab confirmed diagnosis of MCL; (3) have received (a) at least one line of appropriate anti-CD20 based therapy, (b) at least one BTKi therapy, and (4) have had treatment intolerance or failure to the last line of treatment.

Dose and mode of administration: For all patients, antihyperuricemic treatment were given 2-3 days before the first dose of sonrotoclax (i.e., Ramp-up Week 1 Day 1) and continue until ≥7 days after reaching the target daily dose. Allopurinol administered at 300 mg daily was the default prophylaxis treatment for patients unless otherwise noted. Patients allergic to allopurinol must use another uric acid reducer. Sonrotoclax was given to eligible patients at 320 mg once daily starting with a 5-week BIW ramp-up schedule as depicted in Table 1 and FIG. 1, followed by 320 mg of sonrotoclax once per day.

Duration of the treatment: Patients received sonrotoclax until disease progression, unacceptable toxicity, withdrawal of consent, death, loss to follow-up, investigator's decision, or study termination by the sponsor, whichever occurs first.

Criteria for evaluation: The primary efficacy endpoint is the ORR as determined by the Independent Review Committee (IRC).

The main secondary endpoints are: 1) ORR determined by the investigator; 2) DOR determined by the IRC and by the investigator; 3) PFS determined by the IRC and by the investigator; 4) TTR determined by the IRC and by the investigator; and 5) OS as defined as the time from the start of treatment to the date of death due to any cause.

Descriptive statistics were used to analyze all safety data.

Methodology: Assessments of MCL status during the study included disease-related symptoms; targeted physical examination of lymph nodes, liver, spleen, and skin; bone marrow examination (only in case of CR and with bone marrow involvement at baseline); PET-CT and CT scan with contrast of chest, abdomen, pelvis, and neck if clinically indicated; endoscopy if clinically indicated; and PROs. If a CT scan of the neck was performed at baseline and showed evidence of the disease, a CT scan of the neck was required at post-baseline assessments. Response assessments were performed at 8 weeks (study day 56±7 days) and 16 weeks (study day 112±7 days) after the first dose of study drug (i.e., Ramp-up Week 1 Day 1), then every 12 weeks up to one year, then every 24 weeks for 2 years, and yearly thereafter until disease progression, initiation of a new anticancer therapy, withdrawal of consent, death, loss to follow-up, or study termination by the sponsor, whichever came first. The response assessments may be performed within a ±7-day window of the scheduled date.

Safety assessments included adverse events (AEs), serious adverse events (SAEs), clinical laboratory tests, physical examinations, electrocardiograms (ECGs), and vital signs. The investigator assessed the severity for each adverse event and serious adverse event reported during the study. When applicable, adverse events and serious adverse events were assessed and graded based upon the National Cancer Institute Common Terminology Criteria for Adverse Events Version 5.0 (NCI-CTCAE v5.0).

A Safety Monitoring Committee periodically reviewed aggregated safety data from all patients.

### Dose Interruption and Modification

Dosing was held for individual patients for clinically relevant TLS-related laboratory abnormalities, TLS (laboratory or clinical), hematologic toxicities and nonhematologic toxicities, based on investigator assessment of study drug relatedness:

### Hematologic Toxicities

Grade 3 or higher febrile neutropenia
Grade 3 or higher thrombocytopenia that results in clinically significant bleeding
Any ≥Grade 4 hematological toxicity with the following exceptions:
   Grade 4 neutropenia lasting ≤7 days with or without treatment
   Grade 4 lymphopenia
   Grade 4 leukopenia

### Nonhematologic Toxicities:

All instances of hepatic laboratory abnormalities meeting Hy's law criteria
   ALT or AST>3×ULN and total bilirubin>2×ULN without initial findings of cholestasis and after excluding other potential causes.
Any ≥Grade 3 nonhematologic toxicity with the following exceptions:
   TLS-related lab AEs (ie, hyperuricemia, hyperphosphatemia, hyperkalemia, and/or hypocalcemia) that resolve ≤3 days with or without treatment
   Grade 3 gastrointestinal toxicity (ie, nausea, vomiting, diarrhea) unless unresponsive to treatment ≥7 days
   Asymptomatic biochemical lab abnormalities that resolve ≤7 days

Sonrotoclax may be restarted after resolution of the toxicity. If the dose interruption occurred during the ramp-up and lasted ≤7 days, sonrotoclax may be restarted at the same dose level as before the interruption or at 1 dose level lower, per investigator's discretion based on a risk assessment (including tumor burden status). If the dose interruption occurred during the ramp-up and lasted >7 days, the restarting dose must be >1 dose level lower than the last dose given prior to the interruption (Table 4), except for the initial dose of 1 mg.

If dose interruption occurred during the daily dose of 320 mg administration, sonrotoclax may be restarted according to Table 4 and a maximum of 2 dose reductions were allowed.

### Results as of the First DCO Date

Safety: As of the first DCO date, 60 patients with R/R MCL were treated with a 5-week BIW ramp-up schedule followed by 320 mg sonrotoclax once per day. The median age of the patients was 69 years (range: 39 to 85), and 71.7% were male. The median number of prior therapies was 3 (range: 1 to 7). The median duration of treatment with sonrotoclax at the time of data analysis was 7.4 months (range: 0.1 to 13.9 months), 60.0% were exposed for at least 6 months, and 8.3% were exposed for more than 12 months.

TEAEs were reported in 93.3% of patients, and the most frequently reported events (in ≥20% of patients) were anaemia (21.7%) and hyperuricaemia (21.7%). 53.3% of patients reported any TEAE of ≥Grade 3, and the most frequently reported events (in ≥10% of patients) were neutropenia (11.7%) and pneumonia (10.0%). 38.3% of patients reported any SAE. SAEs reported in more than 1 patient were pneumonia (6.7%), acute kidney injury (5.0%).

Approximately a quarter of patients had events leading to dose modifications, with most events leading to dose interruption. Only 1 (1.7%) patients experienced TEAEs leading to dose reduction. The most common TEAEs leading to dose modification were hematologic toxicities and infections, with Pneumonia and Neutropenia (3 patients each, 5.0%) being the most frequently reported events (≥3 patients).

TLS: 5 patients had laboratory TLS, and no clinical TLS was reported. No TLS events resulted in death or treatment discontinuation.

Efficacy: The efficacy results are shown in Table 15-Table 18.

TABLE 15
Analysis of Disease Response by IRC

| | 320 mg QD with 5W |
|---|---|
| Response Category | BIW Ramp-up (N = 60) |
| Best Overall Response, n (%) | |
| CR | 10 (16.7) |
| PR | 26 (43.3) |
| SD | 1 (1.7) |
| PD | 11 (18.3) |
| Could not Be Determined | 12 (20.0) |
| Not Evaluable (NED or Unknown) | 2 (3.3) |
| New Anticancer Therapy before | 4 (6.7) |
| any Disease Assessment | |
| EOS-Death before any Disease | 5 (8.3) |
| Assessment | |
| EOS-Withdrawal By Subject | 1 (1.7) |
| before any Disease Assessment | |
| Overall Response Rate, n (%) | 36 (60.0) |
| 95% CI (%) | 46.5, 72.4 |
| Complete Response Rate, n (%) | 10 (16.7) |
| 95% CI(%) | 8.3, 28.5 |

TABLE 16
Analysis of Disease Response by Investigator

| | 320 mg QD with 5W |
|---|---|
| Response Category | BIW Ramp-up (N = 60) |
| Best Overall Response, n (%) | |
| CR | 11 (18.3) |
| PR | 21 (35.0) |
| SD | 7 (11.7) |
| PD | 16 (26.7) |
| Could Not Be Determined | 5 (8.3) |
| Not Evaluable | 0 (0.0) |
| New Anticancer Therapy before | 1 (1.7) |
| any disease | |
| Assessment | 3 (5.0) |
| EOS-Death before any Disease | |
| Assessment | |
| EOS-Withdrawal By Subject | 1 (1.7) |
| before any Disease Assessment | |
| Overall Response Rate, n (%)^{a} | 32 (53.3) |
| 95% CI (%)^{b} | 40.0, 66.3 |
| Complete Response Rate, n (%) | 11 (18.3) |
| 95% CI (%)^{b} | 9.5, 30.4 |

ªOverall response includes patients who achieve a best overall response of CR or PR.
^{b}The 95% CI was estimated using the Clopper-Pearson method.
^{c}Complete response includes patients who achieve a best overall response of CR.

TABLE 17
Time to Response by IRC

| Time to | 320 mg QD with 5W |
|---|---|
| Response (Months) | BIW Ramp-up (N = 60) |
| n | 36 |
| Mean (SD) | 2.44 (1.130) |
| Median | 1.94 |
| Q1, Q3 | 1.81, 2.63 |
| Min, Max | 1.6, 6.5 |

TABLE 18
Time to Response by Investigator

| Time to | 320 mg QD with 5W |
|---|---|
| Response (Months) | BIW Ramp-up (N = 60) |
| n | 32 |
| Mean (SD) | 2.42 (1.017) |
| Median | 1.94 |
| Q1, Q3 | 1.79, 2.63 |
| Min, Max | 1.6, 6.5 |

### Results as of the Second DCO Date

Safety: As of the second DCO date, for the 60 patients with R/R MCL treated with a 5-week BIW ramp-up schedule followed by 320 mg sonrotoclax once per day, the median duration of treatment with sonrotoclax at the time of data analysis was 7.9 months (range: 0.1 to 19.3 months), 60.0% were exposed for at least 6 months, and 38.3% were exposed for more than 12 months.

TEAEs were reported in 93.3% of patients. Approximately 33.3% (20) of patients had TEAEs leading to dose modifications appearing as dose interruption. No patients had dose reduction. The most common TEAEs were hematologic toxicities, electrolyte abnormalities, and GI disorders. The most common Grade 3+ TEAEs were pneumonia (7 patients, 11.7%) and neutropenia (7 patients, 11.7%).

No major changes were observed by the second DCO date compared to the first DCO date. No new TLS cases were reported since the first DCO date by the second DCO date. No potential Hy's law cases were reported by the second DCO date.

Efficacy: The efficacy results are shown in Table 19-Table 20.

TABLE 19
Analysis of Disease Response by IRC

| | 320 mg QD with |
|---|---|
| | 5W BIW Ramp-up |
| Response Category | (N = 60) |
| Best Overall Response, n (%) | |
| CR | 11 (18.3) |
| PR | 24 (40.0) |
| SD | 2 (3.3) |
| PD | 11 (18.3) |
| Could not Be Determined | 12 (20.0) |
| Overall Response Rate, n (%) | 35 (58.3) |
| 95% CI (%) | 44.9, 70.9 |
| Complete Response Rate, n (%) | 11 (18.3) |
| 95% CI (%) | 9.5, 30.4 |
| Time to Response (Months) | |
| n | 35 |
| Mean (SD) | 2.33 (0.900) |
| Median | 1.94 |
| Q1, Q3 | 1.81, 2.60 |
| Min, Max | 1.6, 6.2 |
| DOR (months) | |
| median (95% CI) | 12.4 |
| | (7.2, NE) |
| Event-free Rate at 6 months, % | 77.2 |
| (95% CI) | (58.0, 88.4) |
| Median follow-up Time (95% CI) | 10.0 |
| (months) | (8.3, 11.8) |
| PFS (months) | |
| median (95% CI) | 9.1 |
| | (4.0, NE) |
| Event-free Rate at 6 months, % | 61.6 |
| (95% CI) | (47.0, 73.3) |
| Median follow-up Time (95% CI) | 12.0 |
| (months) | (9.1, 12.3) |

TABLE 20
Analysis of Disease Response by Investigator

| | 320 mg QD with |
|---|---|
| | 5W BIW Ramp-up |
| Response Category | (N = 60) |
| Best Overall Response, | |
| n (%) | |
| CR | 14 (23.3) |
| PR | 17 (28.3) |
| SD | 8 (13.3) |
| PD | 16 (26.7) |
| Could Not Be Determined | 5 (8.3) |
| Overall Response Rate, n (%)^{a} | 31 (51.7) |
| 95% CI (%)^{b} | 38.4, 64.8 |
| Complete Response Rate, n (%) | 14 (23.3) |
| 95% CI (%)^{b} | 13.4, 36.0 |
| Time to Response (Months) | |
| n | 31 |
| Mean (SD) | 2.29 |
| | (0.701) |
| Median | 1.94 |
| Q1, Q3 | 1.77, 2.60 |
| Min, Max | 1.6, 4.0 |
| DOR by Investigator (months) | |
| median (95% CI) | 13.1 |
| | (6.7, NE) |
| Event-free Rate at 6 months, | 72.0 |
| % (95% CI) | (51.7, 84.9) |
| Median follow-up Time | 10.0 |
| (95% CI) (months) | (9.8, 12.6) |
| PFS by Investigator (months) | |
| median (95% CI) | 12.0 |
| | (9.1, 12.3) |
| Event-free Rate at 6 months, | 61.7 |
| % (95% CI) | (47.9, 72.9) |
| Median follow-up Time | 12.1 |
| (95% CI) (months) | (11.9, 14.5) |

ªOverall response includes patients who achieve a best overall response of CR or PR.
^{b}The 95% CI was estimated using the Clopper-Pearson method.
Complete response includes patients who achieve a best overall response of CR.

### Example 3—Phase ½ Clinical Study to Investigate the Safety of Sonrotoclax Ramp-Up Schedule(s) in Adult Patients with Hematological Malignancies

This is an open-label, global, multicenter, and nonrandomized study to evaluate the safety of novel sonrotoclax dosing and ramp-up schedules with an emphasis on TLS and related toxicity signals.

### Cohort 1 (TN CLL):

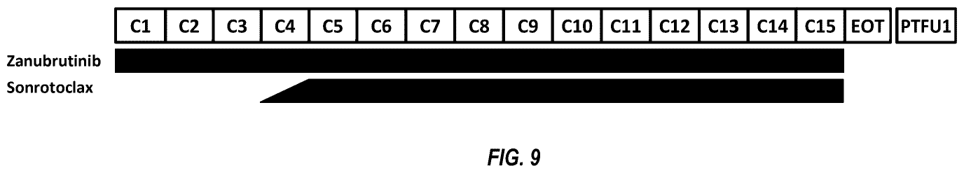

Patient inclusion criteria (Cohort 1): Patients are screened before the treatment. In addition to signed informed consent, each patient eligible to participate in this group must meet all the following inclusion criteria: (1) age 18 years or older; (2) confirmed diagnosis of CLL per the iwCLL criteria; (3) at least 1 measurable lesion based on computed tomography (CT) or magnetic resonance imaging (MRI) per iwCLL; (4) no prior systemic treatment for CLL; and (5) no ongoing clinically significant cardiovascular disease and no active infection including hepatitis B or C. Patients will take zanubrutinib starting on Day 1 of Cycle 1. Zanubrutinib is taken alone daily for first 3 cycles, each cycle lasting 28 days. A TLS risk reassessment will be performed during the third cycle, including tumor clinical assessment, hematology and chemistry laboratories, and CT/MRI scans to guide TLS monitoring and prophylaxis during ramp-up. On Day 1 of Cycle 4, while continuing ongoing zanubrutinib, patients will begin taking sonrotoclax with a ramp-up schedule provided here (e.g., 5-week once-a-week ramp up schedule) to a target dose of 320 mg. Patients will continue to take zanubrutinib and sonrotoclax until the end of the planned total treatment duration of 15 cycles, disease progression, or adverse event requiring discontinuation whichever is early. See FIG. 9.

### Cohort 2 (R/R MCL):

Patient inclusion criteria (Cohort 2): Patients are screened before the treatment. In addition to signed informed consent, each patient eligible to participate in this group must meet all the following inclusion criteria: (1) age 18 years or older; (2) confirmed diagnosis of MCL per World Health Organization (WHO) guidelines; and (3) R/R MCL defined as disease that relapsed after, or was refractory to, at least 1 prior systemic therapy.

Patients will take zanubrutinib starting on Day 1 of Cycle 1. Zanubrutinib is taken alone daily from the first cycle until disease progression, intolerance or adverse event requiring discontinuation. Each cycle is 28 days. A TLS risk reassessment will be performed during the second cycle, including tumor clinical assessment, hematology and chemistry laboratories, and CT/MRI scans to guide TLS monitoring and prophylaxis during ramp-up. On Day 1 of Cycle 2, while continuing ongoing zanubrutinib, patients will begin taking sonrotoclax with a ramp-up schedule provided here (e.g., 5-week once-a-week ramp up schedule) to a target dose of 320 mg. Patients will continue to take sonrotoclax until the end of the planned total treatment duration of 27 cycles, disease progression, intolerance or adverse event requiring discontinuation whichever is early.

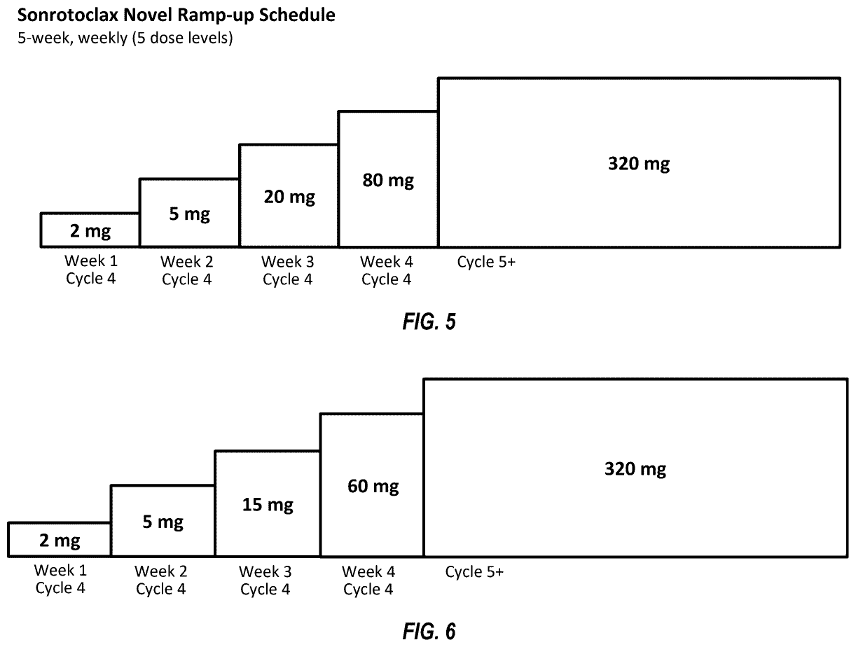

In 5-week once-a-week ramp up schedule, sonrotoclax is ramped up to the target daily dose according to a 5-week once-a-week (Table 21, FIG. 5) ramp-up schedule, which includes associated TLS monitoring and prophylaxis requirements.

TABLE 21
Sonrotoclax Dosing Schedule for 5-Week
Once-A-Week Ramp-up Phase

| Week | Daily Dose |
|---|---|
| Week 1 | 2 mg |
| Week 2 | 5 mg |
| Week 3 | 20 mg |
| Week 4 | 80 mg |
| Week 5 and beyond | 320 mg |

Zanubrutinib is administered orally at a total daily dose of 320 mg per day (either 320 mg once daily or 160 mg twice daily).

The study objective observation period will be the schedule-limiting toxicity (SLT) evaluation window consisting of the first 5 weeks of sonrotoclax treatment encompassing ramp-up+1 week at target daily dose.

### Alternative Novel Ramp-Up Schedules:

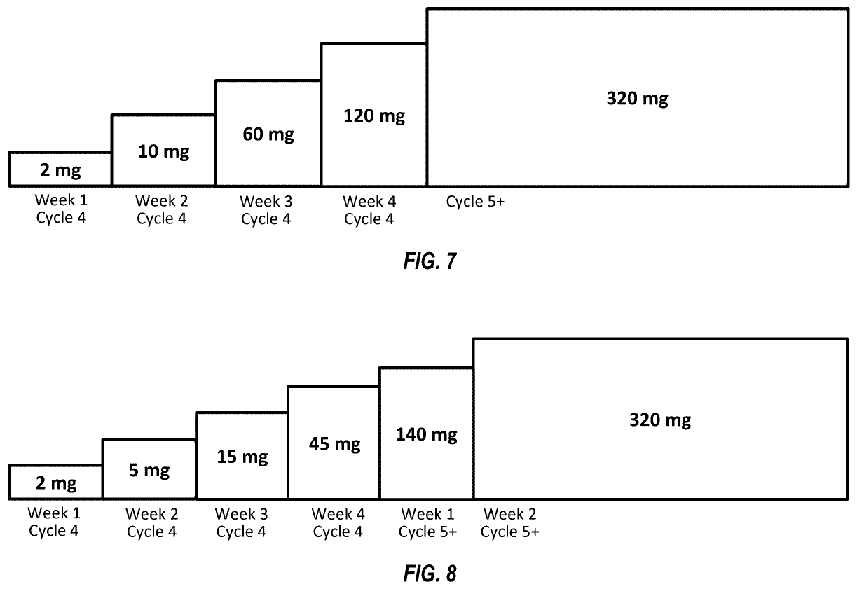

Emergent data may reveal that some modification is needed to an existing schedule. The specific type of modification will depend on the data and will be determined based on SMC recommendation. The SMC may recommend altering aspects of this ramp-up based on emergent data, including ramp-up dose levels or number of ramp-up weeks. In particular, the 5-week once-a-week (Table 21) ramp-up schedule may be replaced by alternative ramp up schedules as recommended by the SMC. Examples of potential alterations and alternatives to 5-week once-a-week ramp up are described in Table 22, Table 23, Table 24, FIG. 6, FIG. 7, and FIG. 8.

The SMC will advise on the use of any alternative schedules as needed, including but not limited to these examples. TLS guidance including prophylaxis, monitoring needs, etc. for ramp-up steps should be identical to those of the 5-week once-a-week ramp up schedule unless otherwise specified.

TABLE 22
Sonrotoclax Dosing Alternative Schedule 1

| Week | Daily Dose |
|---|---|
| Week 1 | 2 mg |
| Week 2 | 5 mg |
| Week 3 | 15 mg |
| Week 4 | 60 mg |
| Week 5 and | 320 mg |
| beyond | |

TABLE 23
Sonrotoclax Dosing Alternative Schedule 2

| Week | Daily Dose |
|---|---|
| Week 1 | 2 mg |
| Week 2 | 10 mg |
| Week 3 | 60 mg |
| Week 4 | 120 mg |
| Week 5 and | 320 mg |
| beyond | |

TABLE 24
Sonrotoclax Dosing Alternative Schedule 3

| Week | Daily Dose |
|---|---|
| Week 1 | 2 mg |
| Week 2 | 5 mg |
| Week 3 | 15 mg |
| Week 4 | 45 mg |
| Week 5 | 140 mg |
| Week 6 and | 320 mg |
| beyond | |

We claim:

1. A method for treating a B-cell malignancy, comprising administering to a patient in need thereof sonrotoclax or a pharmaceutically acceptable salt thereof in therapeutically effective escalating doses followed by a therapeutically effective target daily dose.

2. A method for treating a B-cell malignancy while preventing tumor lysis syndrome (TLS) or reducing a risk of TLS, comprising administering to a patient in need thereof sonrotoclax or a pharmaceutically acceptable salt thereof in therapeutically effective escalating doses followed by a therapeutically effective target daily dose.

3. A method for preventing tumor lysis syndrome (TLS) or reducing a risk of TLS comprising administering to a patient in need thereof sonrotoclax or a pharmaceutically acceptable salt thereof in therapeutically effective escalating doses followed by a therapeutically effective target daily dose, wherein the patient has a B-cell malignancy.

4. A method for treating a B-cell malignancy comprising
administering to a patient in need thereof sonrotoclax or a pharmaceutically acceptable salt thereof in therapeutically effective escalating doses followed by a therapeutically effective target daily dose;
interrupting the administration of sonrotoclax or a pharmaceutically acceptable salt thereof if the patient experiences an adverse reaction; and
restarting the administration of sonrotoclax or a pharmaceutically acceptable salt thereof after the patient is recovered,
wherein:
the adverse reaction is an event suggestive of TLS, a hematological toxicity or a nonhematological toxicity;
when the interruption occurs during the administration of escalating doses,
if the dose interruption lasts up to 7 days, the administration is restarted at the same dose as planned prior to the interruption, and
if the dose interruption lasts greater than 7 days, the administration is restarted at least one dose level lower than the dose as planned prior to the interruption except when the dose as planned prior to the interruption is the initial dose of about 1 mg, then the administration is restarted at about 1 mg; and
when the dose interruption occurs during the administration of the target daily dose,
after the patient is recovered to baseline or Grade 1 toxicity from the first adverse reaction, if the first adverse reaction is an event suggestive of TLS, a hematological toxicity or a Grade 3 nonhematological toxicity, the administration is restarted at the target daily dose, and if the first adverse reaction is a Grade 4 nonhematological toxicity, the administration is restarted at 50% of the target daily dose,
after the patient is recovered from the second and subsequent adverse reactions, the administration is restarted at a daily dose that is 50% of the dose prior to the interruption of the administration, and
if the patient experiences an adverse reaction after two dose reductions, the administration of sonrotoclax or a pharmaceutically acceptable salt thereof is discontinued.

5. The method of any of claims 2-4, where the TLS is clinical TLS.

6. The method of any of claims 2-4, where the TLS is laboratory TLS.

7. The method of any one of claims 1-6, further comprising administering to the patient an antihyperuricemic treatment prior to the administration of the first dose of sonrotoclax or a pharmaceutically acceptable salt thereof.

8. The method of any one of claims 1-7, wherein the B-cell malignancy is chronic lymphocytic leukemia (CLL), small lymphocytic lymphoma (SLL), or mantle cell lymphoma (MCL).

9. The method of any one of claims 1-8, wherein the B-cell malignancy is R/R CLL, R/R SLL or R/R MCL.

10. The method of any one of claims 1-9, wherein the escalating doses comprise a dose of about 1 mg of sonrotoclax per day for half of a week, followed by a dose of about 2 mg of sonrotoclax per day for half a week, followed by a dose of about 5 mg of sonrotoclax per day for half of a week, followed by a dose of about 10 mg of sonrotoclax per day for half of a week, followed by a dose of about 20 mg of sonrotoclax per day for half of a week, followed by a dose of about 40 mg of sonrotoclax per day for half of a week, followed by a dose of about 80 mg of sonrotoclax per day for half of a week, and followed by a dose of about 160 mg of sonrotoclax per day for half of a week.

11. The method of any one of claims 1-10, wherein the escalating doses comprise a dose of about 1 mg of sonrotoclax per day from Week 1 Day 1 to Week 1 Day 3, followed by a dose of about 2 mg of sonrotoclax per day from Week 1 Day 4 to Week 1 Day 7, followed by a dose of about 5 mg of sonrotoclax per day from Week 2 Day 1 to Week 2 Day 3, followed by a dose of about 10 mg of sonrotoclax per day from Week 2 Day 4 to Week 2 Day 7, followed by a dose of about 20 mg of sonrotoclax per day from Week 3 Day 1 to Week 3 Day 3, and followed by a dose of about 40 mg of sonrotoclax per day from Week 3 Day 4 to Week 3 Day 7, followed by a dose of about 80 mg of sonrotoclax per day from Week 4 Day 1 to Week 4 Day 3, and followed by a dose of about 160 mg of sonrotoclax per day from Week 4 Day 4 to Week 4 Day 7.

12. The method of any one of claims 1-9, wherein the escalating doses comprise a dose of about 2 mg of sonrotoclax per day of Week 1, followed by a dose of about 5 mg of sonrotoclax per day of Week 2, followed by a dose of about 20 mg of sonrotoclax per day of Week 3, and followed by a dose of about 80 mg of sonrotoclax per day of Week 4.

13. The method of any one of claims 1-9, wherein the escalating doses comprise a dose of about 2 mg of sonrotoclax per day of Week 1, followed by a dose of about 5 mg of sonrotoclax per day of Week 2, followed by a dose of about 15 mg of sonrotoclax per day of Week 3, and followed by a dose of about 60 mg of sonrotoclax per day of Week 4.

14. The method of any one of claims 1-9, wherein the escalating doses comprise a dose of about 2 mg of sonrotoclax per day of Week 1, followed by a dose of about 10 mg of sonrotoclax per day of Week 2, followed by a dose of about 60 mg of sonrotoclax per day of Week 3, and followed by a dose of about 120 mg of sonrotoclax per day of Week 4.

15. The method of any one of claims 1-9, wherein the escalating doses comprise a dose of about 2 mg of sonrotoclax per day of Week 1, followed by a dose of about 5 mg of sonrotoclax per day of Week 2, followed by a dose of about 15 mg of sonrotoclax per day of Week 3, followed by a dose of about 45 mg of sonrotoclax per day of Week 4, and followed by a dose of about 140 mg of sonrotoclax per day of Week 5.

16. The method of any one of claims 1-15, wherein the therapeutically effective target daily dose is about 320 mg of sonrotoclax or a pharmaceutically acceptable salt thereof.

17. The method of any one of claims 1-16, wherein sonrotoclax or a pharmaceutically acceptable salt thereof is administered once per day.

18. The method of any one of claims 1-17, wherein the administration of sonrotoclax or a pharmaceutically acceptable salt thereof is discontinued if the patient has undetectable minimal residual disease at less than 10⁻⁴ sensitivity (uMRD4) rate.

19. The method of any one of claims 1-18, wherein the administering is discontinued if the patient has undetectable minimal residual disease at less than 10⁻⁶ sensitivity (uMRD6) rate.

20. The method of any one of claims 1-19, wherein sonrotoclax or a pharmaceutically acceptable salt is administered orally.

21. The method of any one of claims 1-20, wherein the antihyperuricemic treatment is administered about two to about three days prior to the administration of the first dose of sonrotoclax or a pharmaceutically acceptable salt thereof.

22. The method of claim 21, wherein the antihyperuricemic treatment is administered about two to about three days prior to the administration of the first dose of sonrotoclax or a pharmaceutically acceptable salt thereof and continues until at least about 7 days after the dose of sonrotoclax or a pharmaceutically acceptable salt thereof reaches the target daily dose.

23. The method of any one of claims 1-22, wherein the patient is an adult who has been previously treated for chronic lymphocytic leukemia (CLL), small lymphocytic lymphoma (SLL), or mantle cell lymphoma (MCL).

24. The method of any one of claims 1-22, wherein the patient is an adult who has been previously treated with a BTK inhibitor.

25. The method of any one of claims 1-22, wherein the patient is an adult who has been previously treated with a BTK inhibitor and anti-CD20 based therapy.

26. The method of any one of claims 1-22, wherein the patient is an adult who has been previously treated with a covalent BTK inhibitor and anti-CD20 based therapy.

27. The method of any one of claims 1-22, wherein the patient is an adult who has been previously treated with a non-covalent BTK inhibitor and anti-CD20 based therapy.

28. The method of any one of claims 1-22, wherein the patient is an adult who has been previously treated with a BCL-2 inhibitor.

29. The method of any one of claims 1-22, wherein the patient is an adult who has been previously treated with a BCL-2 inhibitor and anti-CD20 based therapy.
